SEQUENCE LISTING (10 sequences)

SEQ ID NO: 1 - DNA, 20 bp, Artificial Sequence
  RNA probe sequence obtained from CRISPR design
agaacggtgccatccgccgc

SEQ ID NO: 2 - DNA, 20 bp, Artificial Sequence
  RNA probe sequence obtained from CRISPR design
cgggcgcgtcgaacagctgc

SEQ ID NO: 3 - DNA, 19 bp, Artificial Sequence
  primer alpha-SMA forward
aacacggcatcatcaccaa

SEQ ID NO: 4 - DNA, 18 bp, Artificial Sequence
  primer alpha-SMA reverse
aggcgtagagggaaagca

SEQ ID NO: 5 - DNA, 25 bp, Artificial Sequence
  primer fibronectin forward
cctctgacggcggaacaaacgacca

SEQ ID NO: 6 - DNA, 25 bp, Artificial Sequence
  primer fibronectin reverse
agagggtcccacgttgtactgcttg

SEQ ID NO: 7 - DNA, 24 bp, Artificial Sequence
  primer GAPDH forward
gtgaaggtggagtgaacggatttg

SEQ ID NO: 8 - DNA, 17 bp, Artificial Sequence
  primer GAPDH reverse
tttgatgttggcgggat

SEQ ID NO: 9 - DNA, 7182 bp, Adenovirus Type 9
  repeat_region (1)..(141): inverted terminal repeat region
  promoter (328)..(707): CMV promoter
  promoter (710)..(985): chicken beta-actin promoter
  Intron (986)..(1994): chimeric intron
  gene (2056)..(2121): tPA (tissue plasminogen activator) gene
  gene (2122)..(3555): hMG53 gene
  polyA_signal (3718)..(3773): beta-globin polyA signal
  inverted_terminal_response_vector (4445)..(4585): ITR
  ORIGIN (4647)..(5235): origin sequence
  gene (5406)..(6266): AmpR gene
  promoter (6267)..(6371): AmpR promoter
  origin (6653)..(7108): f1 origin
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcccgcgccattaccctgttatccctaatttaaatctcgatgctaccttaagagaggatctggttctttccgcctcagaagccatagagcccaccgcatccccagcatgctgggagtggcaccttccagggtcaaggaaggcacgggggaggggcaaacaacagatggctggcaactagactcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattccgccaccatggatgcaatgaagagagggctctgctgtgtgctgctgctgtgtggagcagtcttcgtttcgcccatgtcggctgcgcccggcctcctgcaccaggagctgtcctgcccgctgtgcctgcagctgttcgacgcgcccgtgacagccgagtgcggccacagtttctgccgcgcctgcctaggccgcgtggccggggagccggcggcggatggcaccgttctctgcccctgctgccaggcccccacgcggccgcaggcactcagcaccaacctgcagctggcgcgcctggtggaggggctggcccaggtgccgcagggccactgcgaggagcacctggacccgctgagcatctactgcgagcaggaccgcgcgctggtgtgcggagtgtgcgcctcactcggctcgcaccgcggtcatcgcctcctgcctgccgccgaggcccacgcacgcctcaagacacagctgccacagcagaaactgcagctgcaggaggcatgcatgcgtaaggagaagagtgtggctgtgctggagcatcagctggtggaggtggaggagacagtgcgtcagttccggggggccgtgggggagcagctgggcaagatgcgggtgttcctggctgcactggagggctccttggaccgcgaggcagagcgtgtacggggtgaggcaggggtcgccttgcgccgggagctggggagcctgaactcttacctggagcagctgcggcagatggagaaggtcctggaggaggtggcggacaagccgcagactgagttcctcatgaaatactgcctggtgaccagcaggctgcagaagatcctggcagagtctcccccacccgcccgtctggacatccagctgccaattatctcagatgacttcaaattccaggtgtggaggaagatgttccgggctctgatgccagcgctggaggagctgacctttgacccgagctctgcgcacccgagcctggtggtgtcttcctctggccgccgcgtggagtgctcggagcagaaggcgccgccggccggggaggacccgcgccagttcgacaaggcggtggcggtggtggcgcaccagcagctctccgagggcgagcactactgggaggtggatgttggcgacaagccgcgctgggcgctgggcgtgatcgcggccgaggccccccgccgcgggcgcctgcacgcggtgccctcgcagggcctgtggctgctggggctgcgcgagggcaagatcctggaggcacacgtggaggccaaggagccgcgcgctctgcgcagccccgagaggcggcccacgcgcattggcctttacctgagcttcggcgacggcgtcctctccttctacgatgccagcgacgccgacgcgctcgtgccgctttttgccttccacgagcgcctgcccaggcccgtgtaccccttcttcgacgtgtgctggcacgacaagggcaagaatgcccagccgctgctgctcgtgggtcccgaaggcgccgaggcctgatgtacaagtaaagcggccgcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgagctcggtaccaagcttaagtttaaacgctagccatatgaggcctagatctcaattgacgcgtcccggggctaccttaagagagcgcgtatttaaatcgctaccttaggaccgttatagttatcgactgaattggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgcc

SEQ ID NO: 10 - DNA, 6903 bp, Adenovirus Type 9
  repeat_region (1)..(130): inverted terminal repeats
  promoter (155)..(731): CMV promoter sequence
  promoter (777)..(1523): tetON tre3g promoter
  polyA_site (1546)..(1988)
  gene (2005)..(2380): ptre3G gene doxycycline inducible expression
  gene (2397)..(2456): tPA (tissue plasminogen activator) gene
  gene (2465)..(3898): human MG53 gene
  polyA_signal (3929)..(4136): bGH (bovine growth hormone) polyA signal
  repeat_region (4166)..(4306): inverted terminal repeat region
  rep_origin (4381)..(4836): bacteriophafe f1 origin
  gene (5118)..(5222): AmpR gene
  rep_origin (6254)..(6842): origin
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaaactaccggtcgccaccatggctagcatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtaactaagtaaggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctgcaagcctcgtcgtcctggccggaccacgctatctgtgcaaggtccccggccccggacgcgcgctccatgagcagagcgcccgccgccgaggcgaagactcgggcggcgccctgcccgtcccaccaggtcaacaggcggtaaccggcctcttcatcgggaatgcgcgcgaccttcagcatcgccggcatgtccccctggcggacgggaagtatccagctcgacctcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaagtcgagaccggtatggatgcaatgaagagagggctctgctgtgtgctgctgctgtgtggagcagtcttcgtttcgcccgaattcatgtcggctgcgcccggcctcctgcaccaggagctgtcctgcccgctgtgcctgcagctgttcgacgcgcccgtgacagccgagtgcggccacagtttctgccgcgcctgcctaggccgcgtggccggggagccggcggcggatggcaccgttctctgcccctgctgccaggcccccacgcggccgcaggcactcagcaccaacctgcagctggcgcgcctggtggaggggctggcccaggtgccgcagggccactgcgaggagcacctggacccgctgagcatctactgcgagcaggaccgcgcgctggtgtgcggagtgtgcgcctcactcggctcgcaccgcggtcatcgcctcctgcctgccgccgaggcccacgcacgcctcaagacacagctgccacagcagaaactgcagctgcaggaggcatgcatgcgtaaggagaagagtgtggctgtgctggagcatcagctggtggaggtggaggagacagtgcgtcagttccggggggccgtgggggagcagctgggcaagatgcgggtgttcctggctgcactggagggctccttggaccgcgaggcagagcgtgtacggggtgaggcaggggtcgccttgcgccgggagctggggagcctgaactcttacctggagcagctgcggcagatggagaaggtcctggaggaggtggcggacaagccgcagactgagttcctcatgaaatactgcctggtgaccagcaggctgcagaagatcctggcagagtctcccccacccgcccgtctggacatccagctgccaattatctcagatgacttcaaattccaggtgtggaggaagatgttccgggctctgatgccagcgctggaggagctgacctttgacccgagctctgcgcacccgagcctggtggtgtcttcctctggccgccgcgtggagtgctcggagcagaaggcgccgccggccggggaggacccgcgccagttcgacaaggcggtggcggtggtggcgcaccagcagctctccgagggcgagcactactgggaggtggatgttggcgacaagccgcgctgggcgctgggcgtgatcgcggccgaggccccccgccgcgggcgcctgcacgcggtgccctcgcagggcctgtggctgctggggctgcgcgagggcaagatcctggaggcacacgtggaggccaaggagccgcgcgctctgcgcagccccgagaggcggcccacgcgcattggcctttacctgagcttcggcgacggcgtcctctccttctacgatgccagcgacgccgacgcgctcgtgccgctttttgccttccacgagcgcctgcccaggcccgtgtaccccttcttcgacgtgtgctggcacgacaagggcaagaatgcccagccgctgctgctcgtgggtcccgaaggcgccgaggcctgagtcgacctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggacgtacgaagaagcggccgcaggaggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

(19) United States
(12) Patent Application Publication
(10) Pub. No.: US 2020/0179482 A1
(43) Pub. Date: Jun. 11, 2020

(54) COMPOSITION FOR AND METHOD OF FACILITATING CORNEAL TISSUE REPAIR

(71) Applicant: TRIM-EDICINE, INC., COLUMBUS, OH (US)

(72) Inventors: TAO TAN, Columbus, OH (US); HEATHER CHANDLER, Columbus, OH (US); HUA ZHU, Columbus, OH (US); JIANJIE MA, Powell, OH (US)

(73) Assignee: Ohio State Innovation Foundation, Columbus, OH (US)

(21) Appl. No.: 16/598,833

(22) Filed: Oct. 10, 2019

Related U.S. Application Data
(60) Provisional application No. 62/776,839, filed on Dec. 7, 2018.

(51) Int. Cl. A61K 38/17; A61K 9/00; A61K 35/30; C12N 15/86

(52) U.S. Cl. CPC A61K 38/1709; A61K 9/0048; G02B 1/043; C12N 15/86; C12N 2750/14143; A61K 35/30

(57) ABSTRACT

Compositions for and methods of treating corneal injury, among other tissue of or around the eye, are provided. Said compositions comprise MG53 or express MG53. Said compositions can be used for treating chronic or acute injured tissue of the eye or orbit of the eye and can be administered systemically, locally, or both.

### CROSS-REFERENCE TO EARLIER FILED APPLICATION

The present application claims the benefit of provisional application No. 62/776,839, filed Dec. 7, 2018, the entire disclosure of which is hereby incorporated by reference.

### STATEMENT REGARDING FEDERALLY SPONSORED RESEARCH

The U.S. Government has certain rights in this invention pursuant to the following grants. This work was supported by grants from the National Institutes of Health (NIH) to Dr. Hua Zhu (grant No. HL124122) and Dr. Jianjie Ma (grants No. AG056919, No. AR061385, and No. AR070752). This work was also supported by Small Business Innovation Research grants from NIH awarded to Dr. Tao Tan (grant No. GM123887).

### INCORPORATION BY REFERENCE

In compliance with 37 CFR 1.52(e)(5), the instant application contains Sequence Listings which have been submitted in electronic format via EFS and which are hereby incorporated by reference. The sequence information contained in electronic file named TRIM32PRV SEQ ST25.txt, size 22 KB, created on Dec. 7, 2018, using Patent-in 3.5.1, and Checker 4.4.6 is hereby incorporated by reference in its entirety.

### FIELD OF THE INVENTION

The present invention concerns compositions for and methods of facilitating repair of injured tissue of the eye and of the orbit of the eye. It also concerns compositions for and methods of facilitating repair of and preventing undesired fibrosis of and/or vascularization of injured corneal tissue. More particularly, the invention concerns administration of MG53 protein to injured corneal tissue to promote healing thereof or to uninjured corneal tissue to prevent injury thereof.

### BACKGROUND OF THE INVENTION

The corneal endothelium is a single layer of cells on the inner surface of the cornea. It faces the chamber formed between the cornea and the iris. The corneal epithelium is a layer of cells on the outer surface of the cornea, which layer protects underlying corneal stroma from infection, scarring, drying out and other potential harm. The corneal epithelium regenerates itself every one to two weeks. If the corneal surface is irritated or if a section of its epithelial cells erodes away, corneal epithelial stem cells ramp up production to quickly create a new layer of epithelial cells.

The cornea plays an important role in transmitting light and providing protection to the intraocular components of the eye. Due to its exposure to the external environment, the cornea is susceptible to injury and infection. Because the cornea is densely innervated, sustained corneal injury can be painful; delays in repair can increase the risk of corneal scarring and vision loss. Excessive or dramatic injury to corneal tissue can result in infection and scarring leading to partial or complete loss of sight because of the potential for excessive myofibroblast activation and vascular ingrowth which lead to fibrosis and undesired angiogenesis, respectively.

Corneal injury healing is a complex and coordinated process, involving repair to the epithelial layer, migration of viable epithelial cells and fibroblasts for injury closure, and stimulation of cellular proliferation for tissue regeneration. Prevention of excessive stromal myofibroblast activation and vascular in-growth is also imperative to avoid fibrosis and angiogenesis, which can compromise the transparency of the cornea.

The current standard treatment of complicated corneal injury includes maximizing topical lubricants, minimizing evaporative tear loss, using topical antibiotics, protecting the corneal surface with a bandage contact lens, and undergoing surgery. However, even in combination, these measures are often ineffective. Moreover, common side effects of ophthalmic administration of known agents typically include allergic reaction, irritation, itching, swelling, redness of the eye, delayed injury healing, increased pressure in the eye, worsening glaucoma, cataract formation, and injury to the optic nerve. At best, many of these traditional treatments only address one aspect of the corneal healing process.

Autologous serum has been used for treatment of various corneal diseases, and the beneficial effects were largely attributed to growth factors and cytokines. While growth factors can promote healing of the corneal epithelium, they may also have side effects. For example, serum TGF-β may promote fibrotic remodeling of the cornea, and cytokines, such as IL-6, IL-1β, and TNF-α, can cause corneal inflammation.

Although treatment of corneal injuries with specific growth factors and autologous serum may have promise, to date only one biologic (recombinant human neuron growth factor, rhNGF, cenegermin) has been approved for clinical application for promoting epithelial healing. This leaves many clinicians with limited treatment options when dealing with a complicated corneal ulcer and as such, there is an unmet need for therapies to treat corneal injury. Currently, there are no FDA-approved biologic treatments that facilitate corneal injury healing and mitigate scarring.

Under physiologic conditions, limbal (limbus) stem cells (LSC's) participate in injury repair and regeneration of the cornea. Pathologic conditions can disrupt the production of LSC's leading to limbal stem cell deficiency (LSCD). Biological or therapeutic approaches to improve LSC function represent important area of ongoing need in corneal disease and intervention.

MG53 protein (also referred to as mitsugumin 53 or TRIM72) is known in the art: U.S. Pat. No. 7,981,866, WO2008/054561, WO2009/073808, US2011/0202033, US2011/0287004, US2011/0287015, US2013/0123340, WO2011/142744, WO2012/061793, U.S. Pat. Nos. 8,420,338, 9,139,630, 9,458,465, 9,494,602, US2014/0024594, WO2012/134478, WO2012/135868, US2015/0110778, WO2013/036610, US2012/0213737, WO2016/109638, the entire disclosures of which are hereby incorporated by reference.

MG53 is present in serum derived from the blood of mice, rats, and humans (Zhu H, et al., “Amelioration of ischemia-reperfusion-induced muscle injury by the recombinant human MG53 protein” in Muscle & nerve (2015), 52, 852-858; and Liu J, et al., “Cardioprotection of recombinant human MG53 protein in a porcine model of ischemia and reperfusion injury” in Journal of molecular and cellular cardiology (2015), 80, 10-19, the entire disclosures of which are hereby incorporated by reference). Native endogenous LSC's do not express MG53. MG53 and some therapeutic uses thereof are described in the art. It has been thought by artisans in the field of MG53 that it is absent from, meaning it is not endogenous to, the eye, in particular the cornea and aqueous humor.

It would be an important advancement in the art to provide a composition for and method of healing corneal injury that minimizes adverse events associated with conventional drug therapies, reduces corneal scarring, reduces corneal fibrosis, reduces corneal angiogenesis, and/or improves LSC performance, proliferation, and migration.

### SUMMARY OF THE INVENTION

The present invention seeks to overcome some or all of the disadvantages inherent in the art. The present invention provides compositions for and methods of facilitating repair of and preventing fibrosis of and undesired vascularization of injured corneal tissue and of other eye tissue. The present invention results in reduced fibrotic vascularization associated with corneal injury and repair as compared with other known methods of treating corneal injury and as compared to natural healing, meaning healing of corneal tissue in absence of a therapeutic ingredient administered via a pharmaceutical dosage form. Other eye tissues that can be treated include injured tissue of the iris, ciliary body, optic nerve, choroid, sclera, retina, lens, eye socket, orbit of the eye, conjunctiva, limbal tissue, and/or eyelid.

An aspect of the invention provides a method of treating eye injury, the method comprising administering to the injured eye of a subject an effective amount of MG53 in a dosage form. In some embodiments, exogenous MG53 is administered topically to the injured tissue via an ophthalmic dosage form. In some embodiments, MG53 is administered to the subject by way of a dosage form.

An aspect of the invention provides a method of treating corneal injury, the method comprising administering to the injured cornea of a subject an effective amount of MG53 in a dosage form. In some embodiments, exogenous MG53 is administered topically to the cornea via an ophthalmic dosage form.

MG53 is be administered acutely or chronically to treat corneal injury. It can be administered one, two, three or more times per day. It can be administered daily, weekly, monthly, bimonthly, quarterly, semiannually, annually or even longer as needed. It can be administered every other day, five times per week, four times per week, three times per week, two times per week, once daily, twice daily, one to four times daily, continuously, or as frequently or infrequently as needed. The unit dose of each administration is independently selected upon each occurrence from the doses described in this specification or as determined to be therapeutically effective. All combinations of the dosing regimens described are contemplated to be within the scope of the invention.

The dosage forms of the invention can be administered to the eye, the orbit of the eye, tissue adjacent the eye, topically, intramuscularly, intravenously, subcutaneously, subconjunctivally, systemically, or a combination of two or more thereof.

Another aspect of the invention provides an ophthalmic dosage form that releases or provides MG53 into or onto target tissue of the eye. The ophthalmic dosage form can be a non-biological dosage form or a biological dosage form. Suitable dosage forms release or provide MG53 to the surface of the eye, the corneal surface, the surface of the orbit of the eye, the aqueous humor and/or the vitreous humor.

A dosage form can be a liquid, solution, suspension, gel, cream, ointment, implant, explant, slab gel, or coated contact lens.

Another aspect of the invention provides a biological ophthalmic dosage form that releases MG53 or enables expression of MG53 followed by release of MG53 to the cornea or other eye tissue. A biological dosage form is one whose primary carrier or medium or content is a biological product. Suitable biological ophthalmic dosage forms include: a) bioengineered limbal (limbus) stem cells that express and release MG53; b) viral vector, adenoviral vector, or retroviral vector that enters cellular tissue of the eye or eye socket and causes expression of MG53 in said cellular tissue and release of MG53 from said cellular tissue; c) amniotic membrane or amniotic fluid comprising added exogenous MG53; d) autologous blood serum comprising added exogenous MG53; e) collagen shield comprising added exogenous MG53; f) amniotic membrane or amniotic fluid comprising viral vector, adenoviral vector, or retroviral vector that causes expression of MG53 in cellular tissue; g) amniotic membrane or amniotic fluid comprising bioengineered limbal (limbus) stem cells that express and release MG53; h) autologous blood serum comprising viral vector, adenoviral vector, or retroviral vector that causes expression of MG53 in cellular tissue; h) autologous blood serum comprising bioengineered limbal (limbus) stem cells that express and release MG53; i) collagen shield comprising viral vector, adenoviral vector, or retroviral vector that causes expression of MG53 in cellular tissue; j) collagen shield comprising bioengineered limbal (limbus) stem cells that express and release MG53; or k) a combination of any two or more of the above.

The invention provides bioengineered limbal stem cells that express or comprise MG53. The invention also provides a method of converting LSC's that do not express or comprise MG53, otherwise referred to as “non-MG53 LSC's”, to bioengineered LSC's that express or comprise MG53, otherwise referred to as “MG53 LSC's”, the method comprising treating the non-MG53 LSC's with conjugate-labeled MG53, thereby accumulating MG53 in said stem cells to form MG53 LSC's. The invention also provides modified LSC's that comprise exogenously added MG53. The invention also provides a bioengineered stem cell comprising a viral vector comprising a plasmid that induces expression of MG53 in the stem cell.

The invention also provides an autologous serum dosage form comprising exogenously added MG53. The invention also provides an autologous serum dosage form comprising cells that express MG53. The invention also provides an autologous serum dosage form comprising a viral vector that causes cells to express MG53.

The invention also provides a collagen shield dosage form comprising exogenously added MG53. The invention also provides a collagen shield dosage form comprising cells that express MG53. The invention also provides a collagen shield dosage form comprising a viral vector that causes cells to express MG53.

The invention also provides an amniotic membrane dosage form comprising exogenously added MG53. The invention also provides an amniotic membrane dosage form comprising cells that express MG53. The invention also provides an amniotic membrane dosage form comprising a viral vector that causes cells to express MG53.

The invention also provides a coated contact lens dosage form comprising exogenously added MG53. The invention also provides a coated contact lens dosage form comprising cells that express MG53. The invention also provides a coated contact lens dosage form comprising a viral vector that causes cells to express MG53.

The invention also provides a method of converting stem cells (“SC's”) that do not express or comprise MG53, otherwise referred to as “non-MG53 SC's”, to bioengineered SC's that express or comprise MG53, otherwise referred to as “MG53 SC's”, the method comprising treating the non-MG53 SC's with conjugate-labeled MG53, thereby accumulating MG53 in said stem cells to form MG53 SC's. The invention also provides a method of increasing stemness of stem cells, the method comprising treating said stem cells with MG53. The invention also provides a method of protecting stem cells from injury, the method comprising treating said stem cells with MG53. The invention also provides a method of increasing stem cell motility or migration in vivo, the method comprising treating said stem cells with MG53.

The invention also provides a method of preparing a viral vector (VV), otherwise referred to as “VV-MG53”, that induces expression of MG53 in a stem cell (SC), thereby forming a bioengineered SC, otherwise referred to as “VV-MG53-SC”, that expresses or comprises MG53, the method comprising infecting a non-MG53 SC with a VV-MG53. The invention also provides a viral vector (VV), otherwise referred to as “VV-MG53”, that induces expression of MG53 in a stem cell (SC), e.g. limbal stem cell. The invention also provides a viral vector comprising a plasmid that induces expression of MG53 in stem cells following infection of said stem cells with said viral vector.

In some embodiments, the VV-MG53 comprises an adenovirus comprising a plasmid comprising a tissue plasminogen activator (tPA) leader sequence ahead of a human MG53 cDNA, thereby forming a tPA-MG53 sequence. In some embodiments, the plasmid comprises the tPA-MG53 sequence cloned behind a CMV promoter. In some embodiments, the CMV promoter sequence that is controllable via the tetracycline (Tet)-response element (TRE), thereby forming Tet-tPA-MG53 plasmid. In some embodiments, the plasmid further comprises a sequence for SV40-driven transcription of mCherry fluorescent marker. Accordingly, the invention also provides the Tet-tPA-MG53 plasmid, a viral vector comprising the Tet-tPA-MG53 plasmid, and a stem cell comprising the viral vector comprising the Tet-tPA-MG53 plasmid.

The invention provides bioengineered stem cells that express or comprise MG53. In some embodiments, the stem cells comprise a viral vector that causes said stem cells to express MG53, the viral vector-containing stem cells being referred to as “VV-MG53-SC”. In some embodiments, the method of producing VV-MG53-SC comprises the steps of:
constructing a plasmid that induces expression of MG53;
packaging said plasmid in a viral vector; and
infecting stem cells with said viral vector, thereby forming VV-MG53-SC that express MG53.

In some embodiments, a method of producing VV-tPA-MG53-SC comprises the steps of:
constructing a tPA-MG53 plasmid;
packaging said plasmid in a viral vector VV-tPA-MG53; and
infecting stem cells with said viral vector, thereby forming VV-tPA-MG53-SC that express MG53.

In some embodiments, a method of producing VV-tet-tPA-MG53-SC with antibiotic-inducible expression of MG53 comprises the steps of:
constructing a Tet-tPA-MG53 plasmid;
packaging said plasmid in a viral vector VV-tet-tPA-MG53; and
infecting stem cells with said viral vector, thereby forming antibiotic inducible VVtet-tPA-MG53-SC that express MG53.

The invention also provides a method of expressing MG53 in a stem cell, the method comprising:
infecting said stem cell with a viral vector comprising a tPA-MG53 plasmid, thereby causing said stem cell to express MG53.

The invention also provides a method of expressing MG53 in a stem cell, the method comprising:
providing a viral vector comprising a tPA-MG53 plasmid;
infecting said stem cell with said viral vector, thereby causing said stem cell to express MG53.

The invention also provides a method of expressing MG53 in a stem cell, the method comprising:
providing a viral vector comprising a Tet-tPA-MG53 plasmid;
infecting said stem cell with said viral vector VV-tet-tPA-MG53; and
then exposing said stem cell to antibiotic, thereby causing said stem cell to express MG53.

In some embodiments, the plasmid provides inducible expression of MG53 in said stem cells. In some embodiments, the inducible expression is antibiotic-inducible. In some embodiments, the inducible expression is tetracycline-inducible.

In some embodiments, the plasmid comprises a promoter DNA sequence preceding the MG53 DNA sequence. In some embodiments, the plasmid further comprises the TetON DNA sequence preceding the promoter DNA sequence.

Another aspect of the invention provides a cotherapeutic or adjunctive method of treating injured tissue of or around the eye, the method comprising administering to the injured tissue of a subject an effective amount of MG53 and an effective amount of one or more other active ingredients, which are suitable for ophthalmic administration and are efficacious in treating an ophthalmic disease, disorder, injury or condition.

Another aspect of the invention provides a cotherapeutic or adjunctive method of treating corneal injury, the method comprising administering to the injured cornea of a subject an effective amount of MG53 and an effective amount of one or more other active ingredients, which are suitable for ophthalmic administration and are efficacious in treating an ophthalmic disease, disorder, injury or condition. MG53 and said one or more other active ingredients can be administered simultaneous, sequentially or in an overlapping manner.

Another aspect of the invention provides a cotherapeutic or adjunctive method of treating injured tissue of or around the eye, the method comprising administering to a subject in need thereof an effective amount of MG53-expressing stem cells and an effective amount of one or more other active ingredients, which are suitable for ophthalmic administration and are efficacious in treating an ophthalmic disease, disorder, injury or condition. Said MG53-expressing stem cells and said one or more other active ingredients can be administered simultaneous, sequentially or in an overlapping manner.

Another aspect of the invention provides a cotherapeutic or adjunctive method of treating corneal injury, the method comprising administering to a subject in need thereof an effective amount of MG53-expressing stem cells and an effective amount of one or more other active ingredients, which are suitable for ophthalmic administration and are efficacious in treating an ophthalmic disease, disorder, injury or condition. Said MG53-expressing stem cells and said one or more other active ingredients can be administered simultaneous, sequentially or in an overlapping manner.

Another aspect of the invention provides a cotherapeutic or adjunctive method of treating injured tissue of or around the eye, the method comprising administering to a subject in need thereof an effective amount of viral vector that induces MG53-expression in cells, e.g. stem cells, and an effective amount of one or more other active ingredients, which are suitable for ophthalmic administration and are efficacious in treating an ophthalmic disease, disorder, injury or condition. Said viral vector and said one or more other active ingredients can be administered simultaneous, sequentially or in an overlapping manner.

Another aspect of the invention provides a cotherapeutic or adjunctive method of treating corneal injury, the method comprising administering to a subject in need thereof an effective amount of viral vector that induces MG53-expression in cells, e.g. stem cells, and an effective amount of one or more other active ingredients, which are suitable for ophthalmic administration and are efficacious in treating an ophthalmic disease, disorder, injury or condition. Said viral vector and said one or more other active ingredients can be administered simultaneous, sequentially or in an overlapping manner.

Another aspect of the invention provides a cotherapeutic or adjunctive method of treating injured tissue of or around the eye, the method comprising administering to a subject in need thereof an effective amount of antibiotic-inducible viral vector that induces MG53-expression in cells, e.g. stem cells, and an effective amount of one or more other active ingredients, which are suitable for ophthalmic administration and are efficacious in treating an ophthalmic disease, disorder, injury or condition. Said antibiotic inducible viral vector and said one or more other active ingredients can be administered simultaneous, sequentially or in an overlapping manner.

Another aspect of the invention provides a cotherapeutic or adjunctive method of treating corneal injury, the method comprising administering to a subject in need thereof an effective amount of antibiotic-inducible viral vector that induces MG53-expression in cells, e.g. stem cells, and an effective amount of one or more other active ingredients, which are suitable for ophthalmic administration and are efficacious in treating an ophthalmic disease, disorder, injury or condition. Said antibiotic inducible viral vector and said one or more other active ingredients can be administered simultaneous, sequentially or in an overlapping manner.

The invention also provides a method of treating injured tissue of or around the eye, the method comprising administering to a subject in need thereof a viral vector that induces expression of MG53 after administration.

The invention also provides a method of treating corneal injury, the method comprising administering to a subject in need thereof a viral vector that induces expression of MG53 after administration.

The invention also provides a method of treating injured tissue of or around the eye, the method comprising administering to a subject in need thereof stem cells comprising a viral vector that induces expression of MG53 in said stem cells. In some embodiments, the stem cells are LSC's.

The invention also provides a method of treating corneal injury, the method comprising administering to a subject in need thereof stem cells comprising a viral vector that induces expression of MG53 in said stem cells. In some embodiments, the stem cells are LSC's.

The dosage form is independently selected at each occurrence. A combination of two or more different dosage forms can be administered to the subject in need. Two or more different modes of administration can be employed.

Embodiments of the invention exclude compositions comprising single unaltered natural product; however, said compositions may comprise mixtures of said unaltered natural product(s) along with other components thereby resulting in manmade compositions not present in nature. Embodiments of the invention exclude processes that employ solely unaltered natural processes; however, said processes may comprise a combination of said unaltered natural processes along with one or more other non-natural steps, thereby resulting in processes not present in nature. Embodiments of the invention may also include new therapeutic uses (new methods of treatment) for natural products, new compositions comprising said natural products, and new methods employing said natural products.

The invention includes all combinations of the aspects, embodiments and sub-embodiments disclosed herein. Other features, advantages and embodiments of the invention will become apparent to those skilled in the art by the following description, accompanying examples and appended claims.

### BRIEF DESCRIPTION OF THE FIGURES

The following drawings are part of the present specification and are included to further demonstrate certain aspects of the invention. The invention may be better understood by reference to one or more of these drawings in combination with the detailed description of the specific embodiments presented herein.

FIG. 1 depicts before and after images of GFP-MG53 expressed in hCEC's, wherein after injury (righthand figure) the GFP-MG53 has translocated to the mechanical injury site following microelectrode penetration (white arrow).

FIG. 2 depicts a chart of LDH release versus in situ solution concentration of exogenous rhMG53 (μg/ml).

FIGS. 3A and 3B depict images of the cornea of mg53−/− (MG53 knockout; KO; FIG. 3B) and wt (wild-type; FIG. 3A) mice at day-7 following alkaline injury.

FIGS. 3C and 3D depicts charts comparing the vascularization (FIG. 3C) and opacification (FIG. 3D) of the mg53−/− and wt mice following alkaline injury.

FIG. 4 depicts cross-sectional immunofluorescent confocal images of immunohistochemically stained corneas of the mg53−/− and wt mice.

FIG. 5 depicts immunofluorescent confocal images of mg53−/− corneas following alkali injury comparing saline treatment as control versus rhMG53 treatment.

FIG. 6 depicts a charge quantifying the differences in fluorescein uptake of FIG. 5.

FIG. 7 depicts images comparing fluorescein uptake by the alkaline-injured corneal of otherwise healthy rats using saline (as control) and a solution containing rhMG53 in saline.

FIGS. 8A and 8B depict charts comparing the vascularization (FIG. 8A) and opacification (FIG. 8B) of the healthy rats throughout the seven days following alkaline injury and administration of saline (as control) or a solution of rhMG53.

FIG. 9 depicts a confocal microscopic image of corneal fibroblasts that have taken up Alexa647-rhMG53 from extracellular space.

FIG. 10 depicts the generalized construct of the tetON-tPA-MG53 (pAAV9-tetON-tPA-MG53) plasmid of FIG. 12.

FIG. 11 depicts the plasmid map for pAAV9-CAG-tPA-MG53, which comprises 7182 bp.

FIG. 12 depicts the plasmid map for pAAV9-tetON-tPA-MG53, which comprises 6903 bp.

FIG. 13 depicts the DNA sequence (SEQ ID NO. 10) for the pAAV9-CAG-tPA-MG53 plasmid of FIG. 11.

FIG. 14 depicts the DNA sequence (SEQ ID NO. 11) for the pAAV9-tetON-tPA-MG53 plasmid of FIG. 12.

### DETAILED DESCRIPTION OF THE INVENTION

Unless specified otherwise, all embodiments of the invention comprising or employing “MG53” include all known forms of MG53.

As used herein and unless otherwise specified, the term MG53 protein refers to the MG53 protein present as the native form, optimized form thereof, mutant thereof, derivative thereof or a combination of any two or more of said forms. Native MG53 contains 477 amino acids that are well conserved in different animal species. Methods of preparing and/or isolating MG53 are known: U.S. Pat. No. 7,981,866, WO2008/054561, WO2009/073808, US2011/0202033, US2011/0287004, US2011/0287015, US2013/0123340, WO2011/142744, WO2012/061793, U.S. Pat. Nos. 8,420,338, 9,139,630, 9,458,465, 9,494,602, US2014/0024594, WO2012/134478, WO2012/135868, US2015/0110778, WO2013/036610, US2012/0213737, WO2016/109638, the entire disclosures of which, including sequence information therein, are hereby incorporated by reference.

The sequence listing information for native MG53, and variants or various forms thereof, is disclosed in U.S. Pat. Nos. 7,981,866 and 9,139,630, the entire disclosures of which, including sequence information therein, are hereby incorporated by reference. The sequence listing information for a cDNA that encodes optimized native human MG53, or a fragment thereof, is disclosed in U.S. Pat. No. 9,139,630, the entire disclosure of which, including sequence information therein, are hereby incorporated by reference.

As used herein in reference to MG53, the term “mutant” means a recombinant form of MG53 having an amino acid change (replacement) of one, two, three or more amino acids in the amino acid sequence of native MG53. Mutant forms of MG53 and methods of preparing the same are known: US2015/0361146, EP3118317, WO2015/131728, U.S. Pat. No. 9,139,630, the entire disclosures of which, including sequence information therein, are hereby incorporated by reference.

As used herein the term “endogenous MG53”, refers to MG53 present in a subject prior to treatment with a composition, dosage form, or method according to the invention.

The present inventors have discovered that native MG53 protein is present in mammalian corneal epithelia, tear film, and aqueous humor, in particular from the canine or human eye, meaning that native MG53 is endogenous in the eye. The MG53 KO (knockout) mice show reduced expression of ΔNp63α, a marker for LSC's, in the limbus. Following injury, the KO mouse corneas exhibit LSCD (limbal stem cell deficiency) hallmarks with compromised corneal epithelial regeneration, increased goblet cell infiltration, and pronounced stromal fibrosis and vascularization, compared to wild type littermates. The present inventors have also discovered that extracellular MG53, e.g. topically administered MG53 or MG53 released by cells, enters LSC's to improve proliferation and migration thereof under stress conditions. We determined that rhMG53 protein can protect cultured LSC's from injuries in a dose-dependent manner. We conducted a pilot study using an in vivo alkaline-induced injury model in rat corneas and found that rhMG53 (recombinant human MG53) treatment of injured corneas promotes corneal transparency by facilitating injury-repair of the cornea and by reducing post-injury fibrosis and vascularization.

Using semi-quantitative Western blot analysis, the present inventors unexpectedly established the presence of endogenous native MG53 protein (approximately 0.7 ng MG53/mL of aqueous humor) in canine aqueous humor. Moreover, tears obtained from healthy human volunteers were found to contain low levels of endogenous native MG53 protein (at 3.1 ng MG53/mL of tears). Such findings support the potential physiological role of MG53 in corneal injury healing and regeneration. This also supports the safety for the use of rhMG53 protein to treat corneal injury. The inventors, however, also discovered that MG53 is not expressed in native LSC. Accordingly, it was uncertain as to whether LSC's could be successfully modified to express MG53 in vitro and in vivo. The inventors have for the first time successfully prepared bioengineered (genetically modified) LSC's that express MG53.

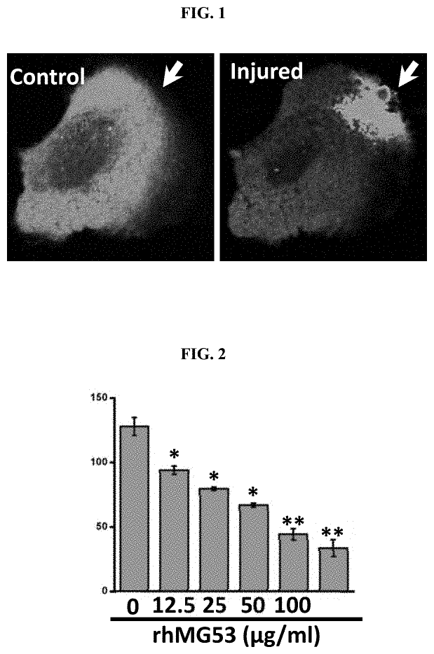

The present inventors established the therapeutic efficacy of expressed MG53 in membrane repair following mechanical injury to the eye. hCEC's (human corneal epithelial cells) were transfected with GFP-MG53 (green fluorescent protein labeled MG53). As depicted in FIG. 1 (left-hand figure), GFP-MG53 expressed in hCEC is localized to the cytosol and intracellular vesicles. In response to injury caused by penetration of a micro-electrode into the membrane, rapid translocation of GFP-MG53 labeled intracellular vesicles towards the injury site was observed (righthand figure). This result suggests that expressed MG53 can be used to affect membrane repair of injured corneal epithelium.

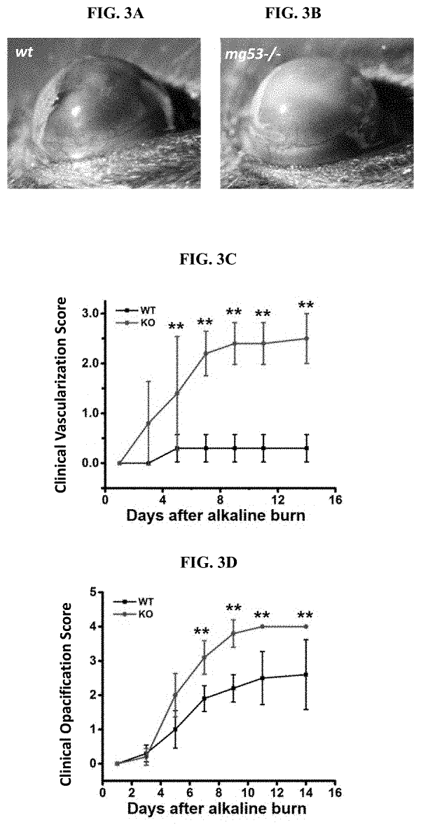

The present inventors established the therapeutic efficacy of exogenous rhMG53 toward protection of hCEC from mechanical injury. hCEC's and micro-glass beads were placed in various solutions containing different concentrations of rhMG53. The glass beads were used to induce injury to the cells following our published procedure discussed in the art cited in the example below. In the absence of rhMG53, the cells exhibit release of high amounts of LDH (lactose dehydrogenase). A reduction in the amount of LDH released as compared to control indicates protection against injury. The injured hCEC's treated with varying doses of MG53 released LDH in a dose-dependent manner, indicating that rhMG53 treatment prevents LDH release following glass bead damage. *: p<0.05; **: p<0.01. The data (FIG. 2) indicate MG53 can be administered exogenously and prophylactically to the eye to minimize injury or damage caused by injury and subsequent poor healing.

Accordingly, the invention provides a method of preventing eye injury, the method comprising administering to a subject in need thereof, one or more dosage forms that provide or induce expression of a prophylactically effective amount of MG53 to the eye. This method is particularly suited for treatment or prevention of chronic eye injury caused by a disease, disorder or condition of the eye.

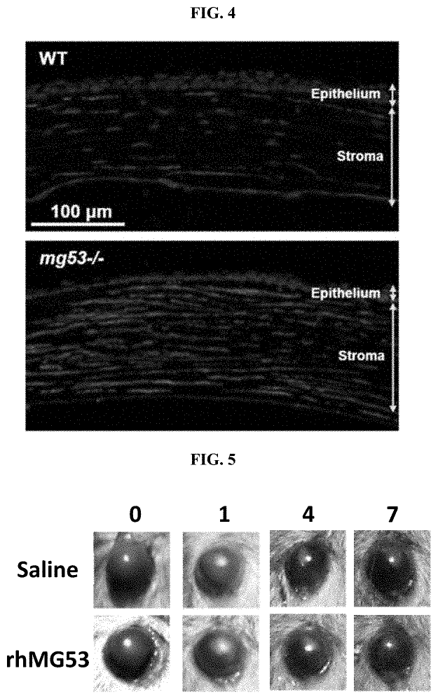

The in vivo efficacy of expressed MG53 toward healing of corneal injury was established using an alkaline solution eye injury model in mg53−/− (MG53 knockout; KO) and wt (wild-type) littermate mice, following published protocols (Anderson C, Zhou Q, Wang S., “An alkali-burn injury model of corneal neovascularization in the mouse” in J. Vis. Exp. (2014) (86), doi. 10.3791/51159). The corneas of mg53−/− (FIG. 3B) and wt (FIG. 3A) mice were injured as described herein, and the extent of vascularization and opacification were determined at day-7 following alkaline injury. A 2-mm filter paper disc soaked in NaOH was applied to the axial cornea for 30 s. Mice were sacrificed 14 days post-injury and pathologic analyses were conducted by a qualified pathologist (in a double blinded manner). In the absence of exogenously administered MG53, the mg53−/− corneas exhibited increased vascularization and opacification as compared to corneas from wt mice. When exogenous MG53 was administered to the injured corneas of the mg53−/− mice, a substantial reduction in vascularization and opacification was observed.

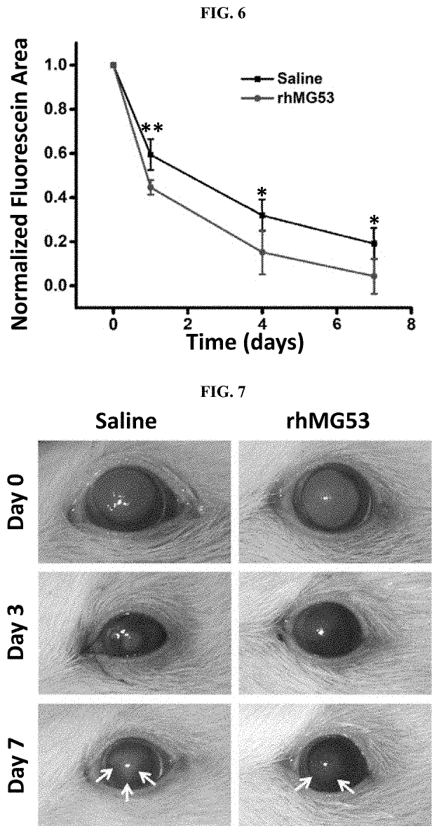

Further proof of the therapeutic efficacy of expressed MG53 toward corneal injury was established by comparing the corneas of mg53−/− and wt mice by performing immunofluorescent confocal imaging of immunohistochemical stained corneas to determine the relative number of epithelial cells. At 14-days post-alkaline injury, globes were fixed either for horizontal sectioning or flat mount staining for histologic evaluation. In the absence of exogenously administered MG53, the injured mg53−/− corneas exhibited significantly fewer epithelial layers than that of wt (3.4±0.6 in wt vs. 2.7±0.6 in mg53−/−, p<0.01) (FIGS. 3C and 3D). We identified the remarkable appearance of conjunctival goblet cells within the corneas of KO mice, but not within the corneas of the WT mice. Such a phenomenon was observed in all examined KO mice (n=5). On average, 14.6±7.1 goblet cells per slide were found in the injured KO cornea. Substantial uveitis was present in the anterior chamber of the KO eye, which reflects potential defects in healing of the injured cornea and inflammation. Corneas derived from the KO mice contained fewer epithelial layers (indicated by DAPI staining of the epithelium, FIG. 4) which is significantly different from that in WT mice following alkaline injury.

Immunohistochemical (IHC) staining with alpha-smooth muscle actin (α-SMA), a specific fibrosis marker, revealed that injured mg53−/− corneas had nearly a five-fold increase in α-SMA expression compared to controls. When flat mount corneas were stained with antibodies against CD31 (a blood vessel marker) and LYVE-1 (a lymphatic vessel marker), we found that the mg53−/− corneas had increased vascular encroachment into the axial cornea (CD31 positive area increased by 40% in mg53−/− corneas as compared to that in wt corneas, p<0.05), further suggesting pronounced angiogenesis in mg53−/− corneas following injury. Accordingly, mg53−/− mouse corneas had significantly fewer epithelial cell layers and more fibrosis (as demonstrated by α-SMA staining) than wt corneas (n=8 per group). *: p<0.05; **: p<0.01.

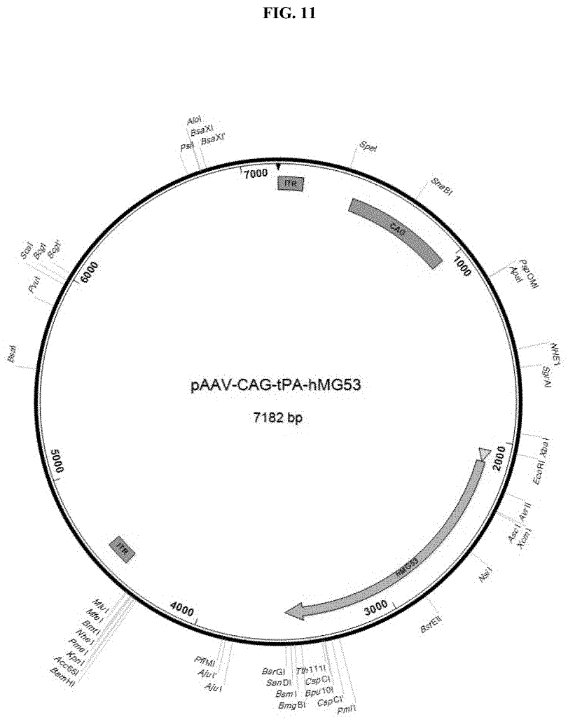

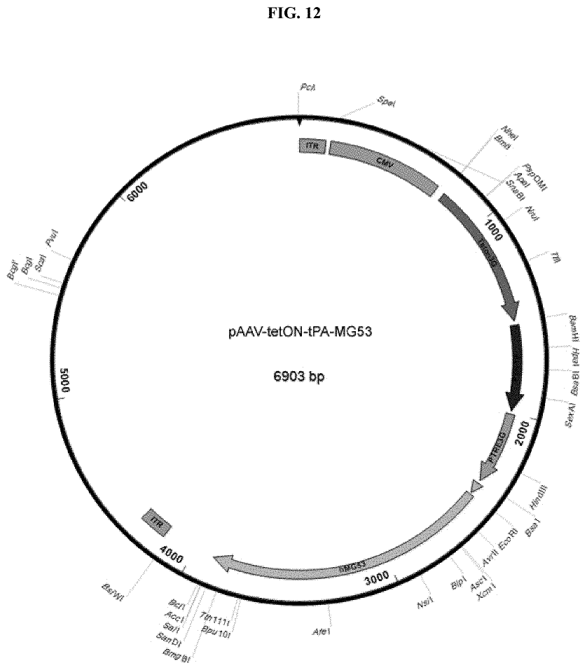

Efficacy of topically administered exogenous MG53 for the healing of corneal injury was evaluated by comparing the vascularization and opacification of alkaline injured corneas in mg53−/− mice and wt rats when administered normal saline solution (NSS; as control) or MG53 in NSS. Following mechanical injury, rats received topical ophthalmic treatment of NSS (as control) or NSS comprising rhMG53 (100 ng of rhMG53/ml of saline; exemplary dosage form of the invention) twice daily for 7 days. The clinical re-epithelialization, fibrotic, and vascularization scores were determined visually (FIG. 7). Exclusion of fluorescein dye was used as an indicator for re-epithelization following injury. We found that topical (exogenous) rhMG53 treatment resulted in healed corneal tissue with retained transparency by facilitating injury-repair of the cornea and reducing post-injury fibrosis and vascularization. Clinical evaluations by an ophthalmologist masked to the treatment found that rhMG53-treated rats had significantly reduced opacification (FIG. 8B) starting at day 6, post alkaline-injury (p<0.05). During the corneal healing process, the rhMG53 treated animals consistently show reduced vascularization (FIG. 8A) scores with a significant difference observed on day 6 (p<0.05).

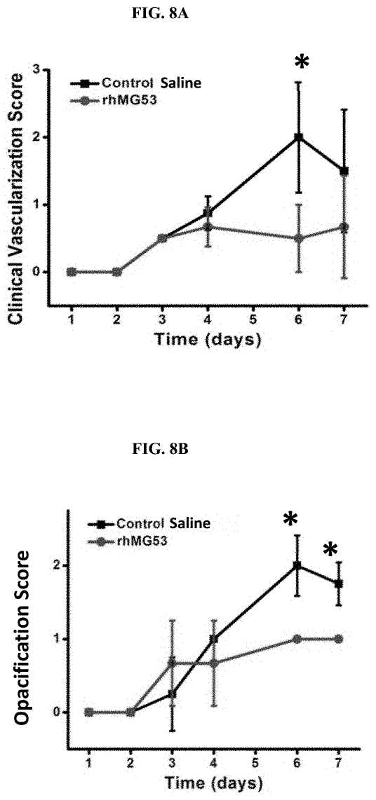

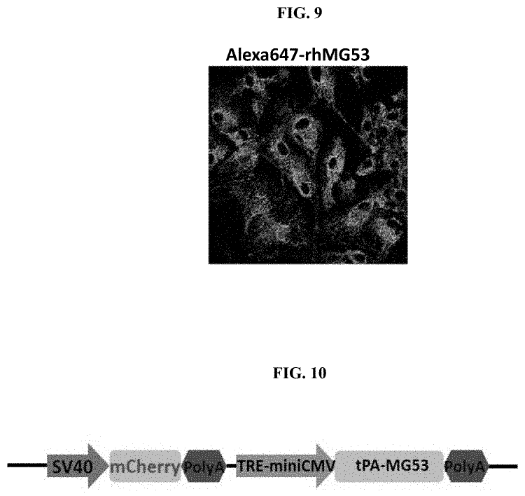

Representative images (FIG. 5) of fluorescein uptake showed that treatment of the injured mg53−/− mouse corneas with rhMG53 (in saline solution containing 100 ng MG53/ml) significantly improved re-epithelialization at 1, 4 and 7 days after alkaline injury as compared to those treated with saline (FIG. 6). Histology was performed on all corneas at the termination of the study. At day 7 post-alkaline injury, reduced fibrosis was also clearly observed in rats that received rhMG53 treatment. A reduction of fibrosis, as compared to control, of about at least 30%, at least 40%, or at least 45% was observed for the MG53 treated corneas.

Evaluation of CD31 expression demonstrated the reduction of superficial and deep vessels in corneal sections derived from rats treated with rhMG53, compared to those receiving saline as control. Expression of α-SMA and CD31 was also significantly reduced in MG53-treated corneas when compared to saline-treated corneas, demonstrating reduced activation of myofibroblasts.

Accordingly, the invention provides a method of and dosage for treating corneal injury. The method comprises administering a therapeutically effective amount of MG53 in one or more dosage forms to the injured corneal. The dosage form comprises a carrier, a tonicity modifier, and MG53. The concentration or amount of MG53 in said dosage form is as described herein. The therapeutically effective amount of MG53 is as described herein.

The inventors have also discovered that MG53 interferes with TGF-β (transforming growth factor β) signaling to control the fibroblast-myofibroblast transition. Excessive TGF-β activation is thought to be one of the underlying causes for the pathogenesis of fibrotic corneal diseases. TGF-β stimulates the transition of the stromal fibroblasts into myofibroblasts, leading to activation of α-SMA and over production of extracellular fibronectin and collagen III.

Serum-starved corneal fibroblasts derived from canines, i.e. corneal tissue that does not express MG53, were treated with TGF-β to induce differentiation into myofibroblasts. Staining with phalloidin revealed the abundant appearance of stress fibers, characteristic of the myofibroblasts. This was further confirmed by WB analysis of α-SMA, a marker of myofibroblasts. Live cell imaging was performed to determine if rhMG53, when added to the extracellular space, could enter the corneal fibroblasts. rhMG53 was conjugated with Alexa647 to allow for live cell imaging under confocal microscopy.

Invitrogen Alexa Fluor 647 (Alexa647) dye is a bright, far-redfluorescent dye with excitation suited for the 594 nm or 633 nm laser lines. Alexa647 is pH-insensitive over a wide molar range. Alexa647 can be conjugated with proteins using a commercial labeling kit (Alexa Fluor™ 647 Protein Labeling Kit Catalog number: A20173, Thermo Fisher Scientific (the method being described in the product manual for the kit) entire disclosure of which is hereby incorporated by reference (Example 4). In brief, 2 mg of lyophilized rhMG53 was diluted in 1 mL diH₂O to a final concentration of 2 mg/mL. rhMG53 (1 mg) was added to a tube containing Alexa 647-NHS ester with sodium bicarbonate to ensure a slightly basic solution. The conjugation mixture was incubated at room temperature for 1 hour while stirring. During the incubation, a resin was loaded into a column and rinsed with PBS. After incubation, the conjugation mixture was gently loaded onto the column and allowed to pass through the resin by adding PBS once the solution was completely within the resin. Conjugated rhMG53-Alexa647 passed through as the bottom band via size exclusion properties of the resin. rhMG53-Alexa647 concentration was determined via Nanodrop spectrophotometry reading at 280 and 650 nm. Calculations for determining concentration are found in the manufacturer's printed protocol.

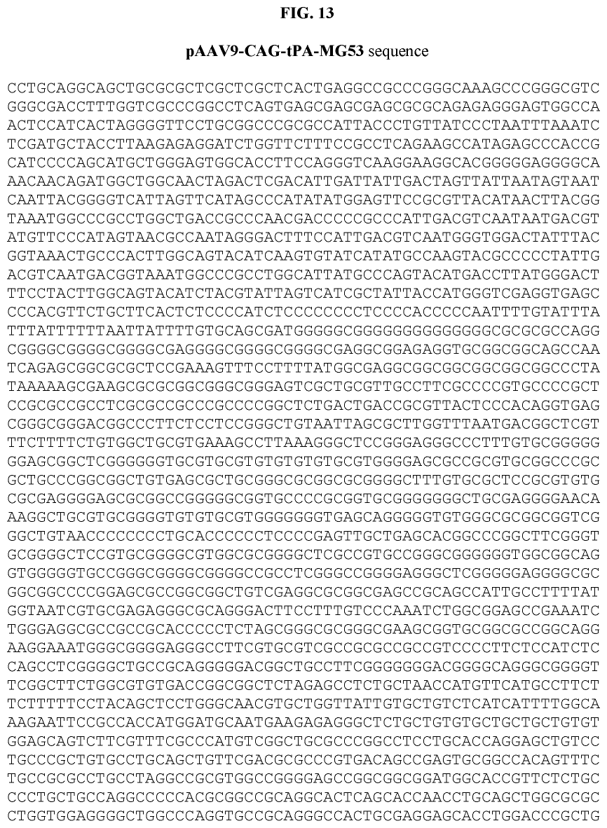

The inventors discovered that Alexa647-rhMG53 could quickly enter the fibroblasts, whereas Alexa647-BSA (as control) failed to penetrate the cells. (FIG. 9) We further used live cell imaging to determine the time course of rhMG53 uptake and found that the entry of rhMG53 occurred within 30 minutes after it was added to the extracellular space.

Accordingly, the invention also provides a conjugate-modified MG53 comprising MG53 conjugated with a fluorescent marker. The fluorescent marker can be a fluorescent protein or a fluorescent dye. Exemplary embodiments include green fluorescent protein (GFP) or Alexa647 fluorescent dye. GFP (described by Prasher et al., “Primary structure of the Aequorea victoria green-fluorescent protein” in Gene. (February 1992) 111(2): 229-33) is a protein composed of 238 amino acid residues (26.9 kDa) that exhibits bright green fluorescence when exposed to light in the blue to ultraviolet range.

In stromal fibroblasts treated with rhMG53, reduced stress fiber formation was observed, together with significant reduction of the mRNA of α-SMA. While TGF-β promoted expression of fibrotic factors, co-treatment of rhMG53 significantly reduced the TGF-β-induced effects. Confocal microscopic imaging revealed that TGF-β treatment induced nuclear translocation of Smad2. The inventors discovered that rhMG53 significantly inhibits nuclear translocation of the Smad2/3 complex, a key event of the canonical TGF-β pathway.

The invention also provides a method of reducing corneal fibrosis and corneal angiogenesis during healing following corneal injury, the method comprising administering to an injured cornea a composition comprising MG53, a carrier, and at least one other pharmaceutically acceptable excipient.

It is important to observe that in wt corneal tissue capable of expressing normal (natural) levels of endogenous MG53, the data herein demonstrate that such level of expression is insufficient to stop eliminate or prevent corneal fibrosis and corneal angiogenesis during healing following corneal injury. It is by administration of exogenous MG53, by way of an ophthalmic dosage form comprising MG53 or causing expression of MG53 or releasing MG53, that corneal fibrosis and corneal angiogenesis can be prevented, reduced, or eliminated. It is also by administration of MG53, by way of a bioengineered ophthalmic dosage form comprising MG53 or expressing MG53, that corneal fibrosis and corneal angiogenesis can be prevented, reduced, or eliminated.

mg53−/− mice show reduced expression of ΔNp63a in the limbus, a marker for LSC's suggesting the LSC population in KO mice may be compromised. To investigate the potential role of MG53 on LSC's, we adopted an established protocol to isolate LSC from the mouse limbus. Identity and purity of the isolated LSCs were confirmed by immunostaining, where >95% of the LSC's were positive for ΔNp63a and negative for staining with vimentin, a marker for stromal fibroblasts. Based on immunostaining and WB (Western blot) analyses, we found that LSC's do not contain endogenous MG53 protein. This absence of MG53 expression in LSC's means that the eye is naturally deficient in repairing corneal injury. According to the invention, administration of exogenous MG53 or administration of LSC's that express MG53 or administration of viral vectors that cause expression of MG53 in cells can provide substantial improvement in the healing process of corneal injury.

According to one embodiment, LSC's that express MG53 were prepared by using Alexa 647-rhMG53. Using live cell imaging with fluorescent-labeled rhMG53, we determined that Alexa 647-rhMG53 could rapidly enter LSC's, whereas Alexa 647-BSA (as control) could not. Accordingly, the invention also provides a method of converting LSC's that do not express MG53 into LSC's that do express MG53, the method comprising treating LSC's that do not express MG53 with fluorescent-labeled rhMG53, thereby forming said LSC's into LSC's that do express MG53. The invention also provides modified, e,g, bioengineered, LSC's that express MG53. The invention also provides modified LSC's that comprise MG53. The invention also provides modified LSC's that comprise exogenously added MG53.

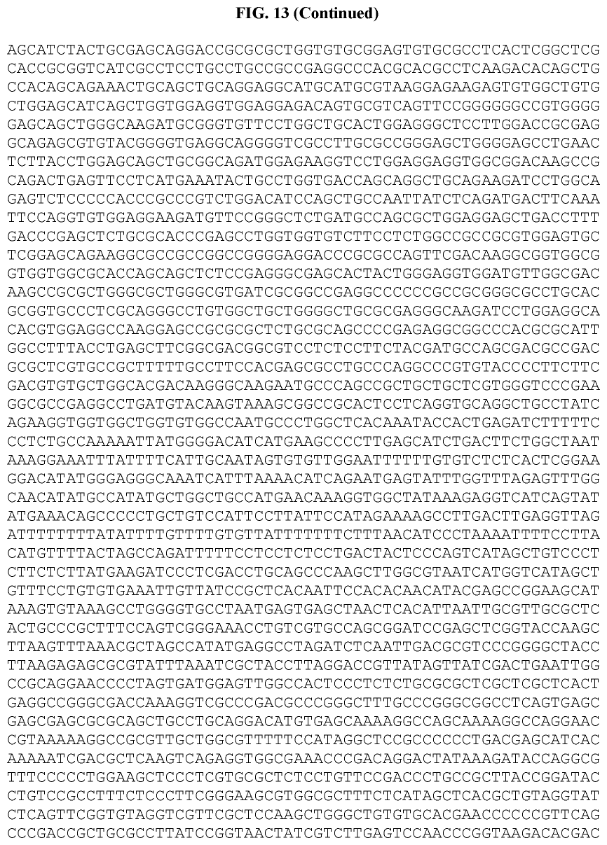

According to another embodiment, modified LSC's that express MG53 were prepared by infecting the LSC's that do not express MG53 with an adenovirus vector (AAV) containing the plasmid pAAV-tet0N-tPA-MG53 and treating the genetically modified LSC's with antibiotic, e.g. doxycycline or tetracycline, to induce expression of MG53 in the LSC's (Examples 13 and 14). We constructed a plasmid with inducible secretion of MG53 by adding a tissue plasminogen activator (tPA) leader sequence ahead of the human mg53 cDNA. The tPA-MG53 sequence was cloned behind a minimum CMV promoter that is under the control of a tetracycline response element (TRE). This plasmid also contained a SV40-driven transcription of mCherry fluorescent marker, allowing for visualization and selection of transfected cells (FIG. 10 depicts the generalized construct of the pAAV-tet0N-tPA-MG53 plasmid).

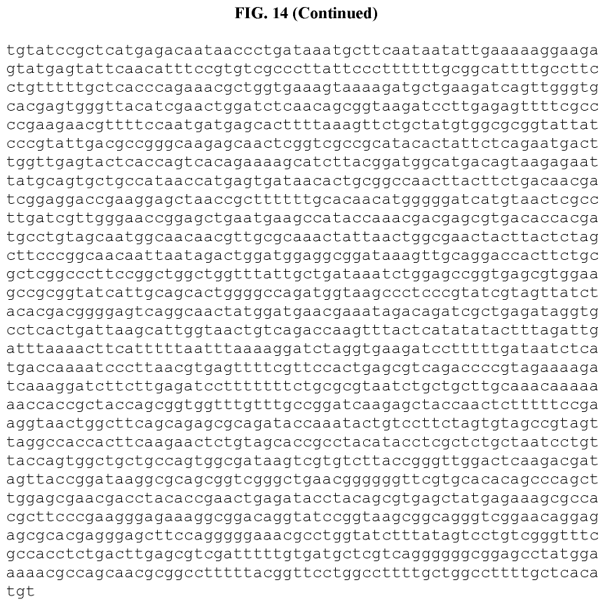

This TetON-tPA-MG53 plasmid (otherwise known as the pAAV-tet0N-tPA-MG53 plasmid) was packaged into the adenovirus for efficient infection of the LSC's. After infection (24 h), bioengineered LSC's were harvested for WB assay. Treatment of the LSC's with increasing doses of doxycycline (Dox) led to elevated secretion of MG53 into the culture medium, as well as intracellular MG53 expression in the LSC's. Quantitative assessment with WB and ELISA demonstrated that 0.02-0.2 pg MG53 protein/cell could be achieved with tPA-MG53 in LSC's. FIG. 14 depicts the gene sequence of the pAAV9-TetON-tPA-MG53 plasmid.

Accordingly, the invention provides a viral vector comprising a plasmid that induces expression of MG53 in stem cells following infection of said stem cells with said viral vector.

The invention also provides a bioengineered stem cell comprising a viral vector comprising a plasmid that induces expression of MG53 in the stem cell.

The ability of MG53 to protect LSC's from mechanical injury was established using the glass bead evaluation described herein. When rhMG53 was applied in varying concentrations to the LSC's in culture medium, a dose-dependent effect was observed in which rhMG53 could protect against mechanical injury to the cultured LSC's. This protective effect is observed in bioengineered LSC's of the invention as well as native (non-bioengineered) LSC's. Moreover, using a colony formation assay, rhMG53's role in regulating the sternness of LSC's was evaluated. LSC's treated with rhMG53 showed a significant increase in colony forming units, suggesting enhanced sternness of the LSC's. rhMG53 enhanced proliferation of LSC's under normal culture conditions.

The invention thus provides a method of increasing sternness of stem cells, the method comprising treating said stem cells with MG53. The invention also provides a method of protecting stem cells from injury, the method comprising treating said stem cells with MG53.

Further proof of efficacy of MG53 toward treating corneal injury was obtained using a scratch injury assay. The assay was performed using LSC's and rhMG53 in the assay medium. We discovered that treatment of the LSC's with rhMG53 improved migration of LSC's in a dose dependent manner, in particular for solutions comprising at least 25 jag of MG53/ml, or at least 50 μg of MG53/ml.

The invention thus provides a method of increasing stem cells motility or migration in vivo, the method comprising treating said stem cells with MG53.

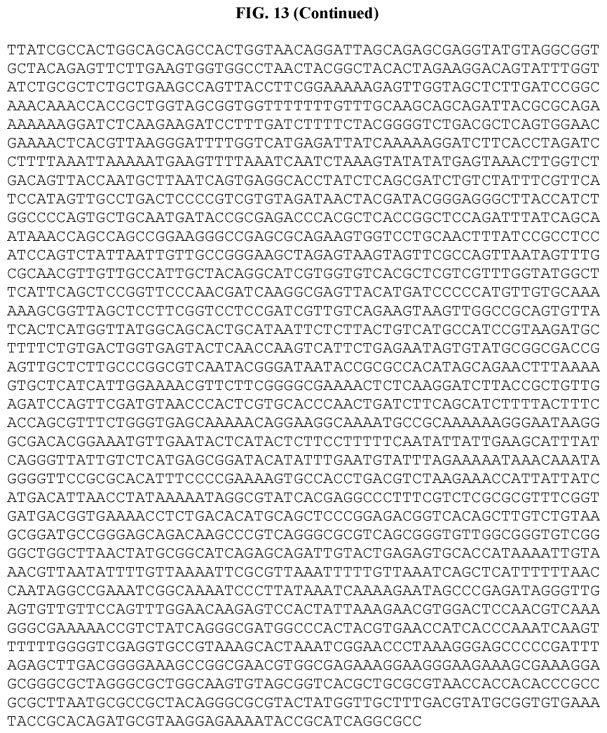

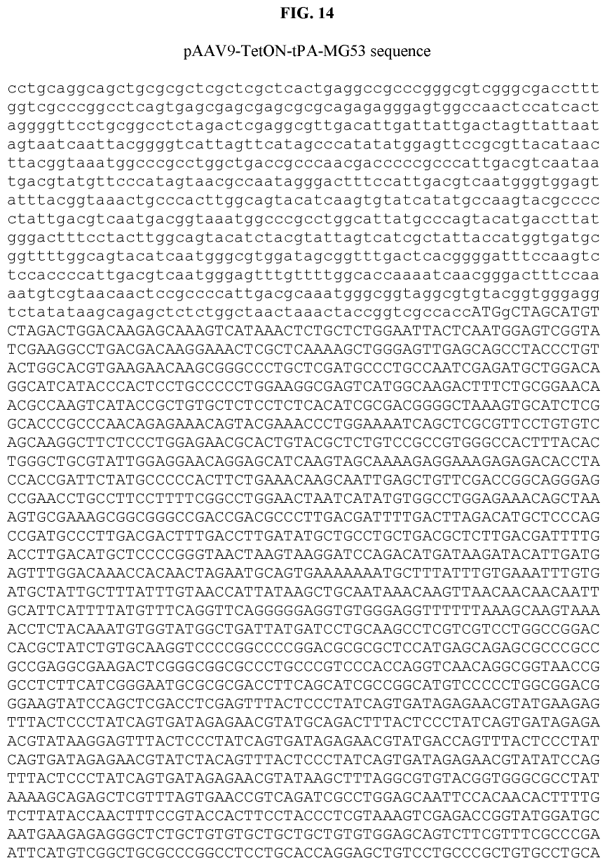

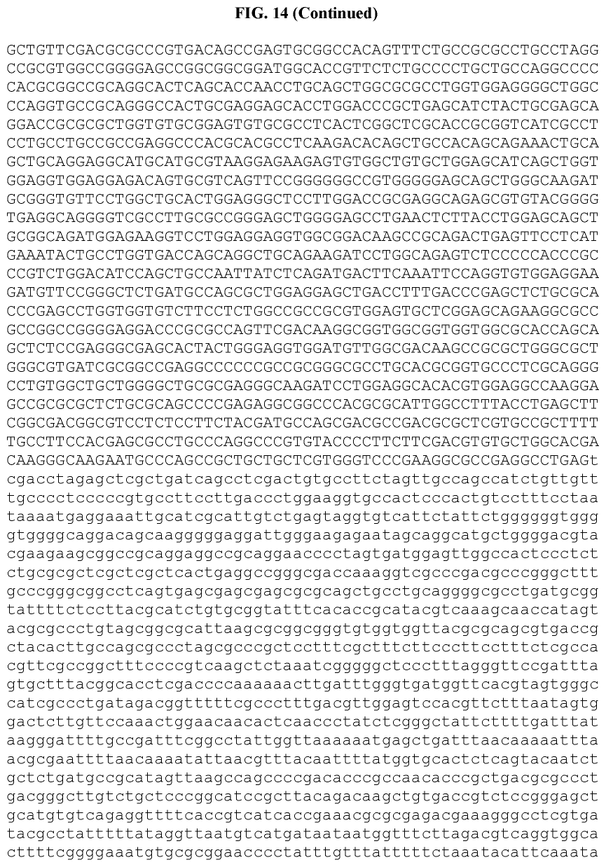

The inventors have also discovered that subconjunctival administration of pAAV-tPA-MG53 resulted in substantial expression of MG53 in tears. tPA-hMG53 (hMG53 refers to human MG53) or Tet-tPA-MG53 were packed into AAV type 9 (adenovirus type 9) to produce pAAV9-tetON-tPA-MG53 (TetON refers to the well-known tetracycline inducible promoter) and pAAV9-CAG-tPA-MG53 (CAG refers to the well-known CAG promoter; a constitutive promoter). The gene sequence of the pAAV9-tetON-tPA-MG53 plasmid is depicted in FIG. 14 and its plasmid map is depicted in FIG. 12. The gene sequence of the pAAV9-CAG-tPA-MG53 plasmid is depicted in FIG. 13 and its plasmid map is depicted in FIG. 11.

The most important motifs of the pAAV9-tetON-tPA-MG53 (SEQ ID NO. 11) plasmid are as follows:

| Base No. | Motif |
|---|---|
| 1-130 | ITR (inverted terminal repeats) |
| 155-731 | CMV promoter |
| 777-1523 | Teton tre3g promoter |
| 1546-1988 | poly A |
| 2005-2380 | PTRE3G (Vector for doxycycline-inducible expression) |
| 2397-2456 | tPA |
| 2465-3898 | hMG53 |
| 3929-4136 | bGH polyA Signal |
| 4166-4306 | ITR |
| 4381-4836 | f1 ori |
| 5118-5222 | AmpR |
| 6254-6842 | ori |

The most important motifs of the pAAV9-CAG-TPA-MG53 (SEQ ID NO. 10) plasmid are as follows:

| Base No. | Motif |
|---|---|
| 1-141 | ITR |
| 328-707 | CMV enhancer |
| 710-985 | Chicken β-actin promoter |
| 986-1994 | Chimeric Intron (for enhancing transgene expression) |
| 2056-2121 | tPA |
| 2122-3555 | hMG53 |
| 3718-3773 | β-globin Poly(A) |
| 4445-4585 | ITR |
| 4647-5235 | Ori |
| 5406-6266 | AmpR |
| 6267-6371 | AmpR promoter |
| 6653-7108 | f1 Ori |

Mice were injected with 10 μl virus per eye at subconjunctiva or cornea (the titer of the AAV viruses was about 5.0×10¹²). In mice injected with pAAV9-tetON-tPA-MG53, doxycycline was administered at a dose of 2.5 mg/kg per day intraperitoneally for two weeks. 14 days post injection of pAAV9-tPA-MG53, IHC staining of the mouse corneal demonstrated the presence of MG53 in corneal tissue. By way of Western blot, we determined that Dox-inducible MG53 secretion was observed in the mouse tears and cornea at 14 days after subconjunctival administration of pAAV9-tetON-tPA-MG53.

Accordingly, the invention provides a method of treating corneal injury or eye injury, the method comprising the step of administering to the eye of a subject in need thereof an viral vector that induces expression of MG53 in therapeutically effective amounts. The invention also provides a method of increasing the expression of MG53 in tears, the method comprising the step of administering to the eye of a subject in need thereof a viral vector that induces expression of MG53 in therapeutically effective amounts. The methods can alternatively or additionally comprise the step of administering to the subject stem cells that express MG53. Subconjunctival administration, intraocular administration, intraorbtal administration, and topical administration or combinations thereof are particularly suitable.

Suitable concentrations of MG53 in a dosage form include at least 1 ng of MG53/ml, at least 5 ng of MG53/ml, at least 10 ng of MG53/ml, at least 25 ng of MG53/ml, at least 50 ng of MG53/ml, at least 75 ng of MG53/ml, at least 100 ng of MG53/ml, at least 250 ng of MG53/ml, at least 500 ng of MG53/ml, at least 750 ng of MG53/ml, at least 1 μg of MG53/ml, at least 5 μg of MG53/ml, at least 10 μg of MG53/ml, at least 15 μg of MG53/ml, at least 20 μg of MG53/ml, at least 25 μg of MG53/ml, at least 30 μg of MG53/ml, at least 50 μg of MG53/ml, or at least 100 μg of MG53/ml. Higher concentrations are also acceptable, particularly in view the efficacy dose-response trend observed for MG53. These doses can be administered on a frequency as described herein or as determined to be most effective.

Suitable doses of MG53 that can be administered to a subject in one or more dosage forms include at least 1 ng of MG53, at least 5 ng of MG53, at least 10 ng of MG53, at least 25 ng of MG53, at least 50 ng of MG53, at least 75 ng of MG53, at least 100 ng of MG53, at least 250 ng of MG53, at least 500 ng of MG53, at least 750 ng of MG53, at least 1 μg of MG53, at least 5 μg of MG53, at least 10 μg of MG53, at least 15 μg of MG53, at least 20 μg of MG53, at least 25 μg of MG53, at least 30 μg of MG53, at least 50 μg of MG53, or at least 100 lag of MG53. Such doses can be on a total body weight basis or a per kg of body weight basis.

Some embodiments of the invention provide a method for treating eye injury by increasing expression of or overexpressing MG53 in tissue surrounding the eyeball, i.e. any tissue defining the eye socket, such that MG53 is released into the eye socket and onto the eye ball, including the cornea. As evidence of efficacy, an established tPA-MG53 transgenic mouse model was used. The tPA-MG53 mice express high levels of circulating MG53, i.e. systemic endogenous MG53. Corneal flat mounts derived from tPA-MG53 mice were probed with antibody against ΔNp63a to stain for LSC's. Compared with WT mice, increased intensity of ΔNp63a was observed and WB confirmed enhanced protein levels of ΔNp63α. Moreover, increased IHC staining of MG53 in the limbus and corneal epithelium was observed in tPA-MG53 mice, indicating that elevated levels of MG53 in circulation leads to accumulation of MG53 in the limbus and cornea.

The data establish that systemically overexpressed MG53 in tissue surrounding the eyeball will be useful for the treatment of eye injury, in particular corneal injury. This is because the tissue defining the orbit of the eye releases MG53 into the orbit and thus onto the surface of the eye, including corneal surface. Likewise, tears of tPA-MG53 mice also contain raised levels of MG53, so, increased levels of MG53 are released into the orbit of the eye and onto the surface of the eye.

Accordingly, the invention also provides a method of treating eye injury by systemically or locally administering to a subject in need thereof a bioengineered cell (such as a LSC) and/or a bioengineered viral vector (such as a retroviral vector) to cause increased expression of MG53 in the eye or eye socket of said subject. Following administration to the subject, the LSC will express MG53 in the eye or eye socket of said subject. Likewise, the viral vector will either express or induce expression of MG53 in the eye or tissue of eye socket of said subject. The bioengineered LSC and/or viral vector may be administered to the eye, the orbit of the eye, tissue adjacent the eye, intramuscularly, intravenously, subcutaneously, subconjunctivally, or systemically.

The inventors also discovered that long term expression of MG53 in the eye or tissue of the orbit is not harmful. Lineage tracing experiments using the K14ER^{T2}/R26R-Confetti mice were conducted. R26R-Confetti mice were purchased from Jackson Laboratories and crossed with our tPA-MG53 mice, subsequently generating tPA-MG53/K14ER^{T2}/R26R-Confetti mice. Hot DMSO (60° C.) was used to induce cornea injury. Tamoxifen supplemented in DMSO (200 mg/mL) was used to activate Cre-recombinase to drive fluorescent protein expression in LSC's positive for K14. More activated LSC's were observed in mice harboring the tPA-MG53 background than those in the WT control. This data provide evidence of MG53's role in modulating LSC activation in a physiological setting. Analysis of corneal morphology of aged tPA-MG53 and WT mice (30 months old), revealed no visible pathology in the eye, and age-related stromal thinning was less pronounced in tPA-MG53 corneas compared to WT. The data indicate that the sustained elevation of MG53 in the cornea is safe.

The amount of therapeutic compound (MG53) incorporated in each dosage form will be at least one or more unit doses and can be selected according to known principles of pharmacy. An effective amount of therapeutic compound is specifically contemplated. By the term “effective amount”, it is understood that, with respect to, for example, pharmaceuticals, a pharmaceutically (therapeutically) effective amount is contemplated. A pharmaceutically effective amount is the amount or quantity of a drug or pharmaceutically active substance which is sufficient to elicit the required or desired therapeutic response, or in other words, the amount which is sufficient to elicit an appreciable biological response when administered to a patient.

The term “unit dosage form” is used herein to mean a dosage form containing a quantity of the drug, said quantity being such that one or more predetermined units may be provided as a single therapeutic administration.

The dosage form is independently selected at each occurrence from the group consisting of liquid solution, suspension, gel, cream, ointment, slab gel, insert (implant).

The dosage form can also include collagen shield, amniotic membrane, autologous blood serum, and/or coated contact lens.

Exemplary ophthalmic dosage forms are disclosed by Baranowski et al. (Ophthalmic Drug Dosage Forms: Characterization and Research Methods” in Sci. World J. (2014), Article ID 861904, pp 1-14, http://dx.doi.org/10.1155/2014/861904), Bourlais et al. (Ophthalmic Drug Delivery Systems—recent advance” in Prog. Retin. Eye Res. (1998), 17(1), 33-58), Del Amo et al. (“Current and Future ophthalmic drug delivery systems, A shift to the posterior segment” in Drug Disc. Today (2008), 13(3-4), 135-143), Conway et al. (“Recent patents on ocular drug delivery systems” in Recent Pat. Drug Deliv. Formul. (2008), 2(1), 1-8); Abdelkader et al. (“Controlled and continuous release ocular drug delivery systems: pros and cons” in Curr. Drug Deliv. (2012), 9(4), 421-430), Destruel et al. (In vitro and in vivo evaluation of in situ gelling systems for sustained topical ophthalmic delivery: state of the art and beyond” in Drug Discov. Today (2017), 22(4), 638-651), Achouri et al. (“Recent advances in ocular drug delivery” in Drug Dev. Ind. Pharm. (2013), 39(11), 1599-1617), and Addo (“Ocular drug delivery: advances, challenges and applications” (ed. RT Addo; Springer (2016), pp 1-185 Cham, Switzerland), the entire disclosures of which are hereby incorporated by reference.

Dosage forms comprising autologous (blood) serum can be made as described by Geerling et al. (“Autologous serum eye drops for ocular surface disorders” in British Journal of Ophthalmology (2004) 88:1467-1474; http://dx.doi.org/10.1136.bjo.2004.044347) or by Fox et al. (Beneficial effect of tears made with autologous serum in patients with keratoconjunctivitis sicca in Arthritis Rheum. (1984), 28:459-461), the entire disclosures of which are hereby incorporated by reference, or as described herein (Example 16). In some embodiments, exogenous MG53 is added to the dosage forms, or stem cells expressing MG53 are added to the dosage forms, or viral vectors that cause cells to express MG53 are added to the dosage forms, or embodiments of two or more such systems are employed in said dosage form(s).

Accordingly, the invention provides an autologous serum dosage form comprising exogenously added MG53. The invention also provides an autologous serum dosage form comprising cells that express MG53. The invention also provides an autologous serum dosage form comprising a viral vector that causes cells to express MG53.

Dosage forms comprising a collagen shield can be made as described herein (Example 18). In general, collagen shields are manufactured from porcine scleral tissue or bovine corium (dermis) collagen and contain mainly type I collagen and some type III collagen. They are shaped like a contact lens and are supplied in a dehydrated form, requiring rehydration prior to insertion. Variations in collagen crosslinking can be induced with ultraviolet light (UV) during manufacture dictate lens duration before dissolution. Three different collagen shields are currently available with dissolution times of 12, 24, and 72 hours. Corneal collagen shields have a diameter of 14.5-16.0 mm, a base curve of 9 mm, and a central thickness of 0.15-0.19 mm. A study of the oxygen transmissibility of Bio-Cor (Bausch and Lomb, Clearwater, Fla.) collagen shields in vitro indicated that they behave like a 63% water-content hydrogel contact lens with an average oxygen permeability (Dk/L) of 27 1011 cm² mL 02/s mL mm Hg. Water-soluble compounds are trapped within the collagen matrix and some drugs undergo reversible binding to collagen. The collagen shield becomes saturated when placed in an aqueous solution comprising MG53; greater drug absorption occurs when the shield is soaked with higher protein concentrations. Therefore, the collagen shields can be soaked in saline solution containing rhMG53 protein (100 ng/ml). In some embodiments, exogenous MG53 is added to the dosage forms, or stem cells expressing MG53 are added to the dosage forms, or viral vectors that cause cells to express MG53 are added to the dosage forms, or embodiments of two or more such systems are employed in said dosage form(s).

Accordingly, the invention provides a collagen shield dosage form comprising exogenously added MG53. The invention also provides a collagen shield dosage form comprising cells that express MG53. The invention also provides a collagen shield dosage form comprising a viral vector that causes cells to express MG53.

Dosage forms comprising an amniotic membrane can be made as described herein (Example 17). In some embodiments, exogenous MG53 is added to the dosage forms, or stem cells expressing MG53 are added to the dosage forms, or viral vectors that cause cells to express MG53 are added to the dosage forms, or embodiments of two or more such systems are employed in said dosage form(s).

Accordingly, the invention provides an amniotic membrane dosage form comprising exogenously added MG53. The invention also provides an amniotic membrane dosage form comprising cells that express MG53. The invention also provides an amniotic membrane dosage form comprising a viral vector that causes cells to express MG53.

Dosage forms comprising a coated contact lens can be made as described herein (Example 20) or as described by Bobba et al. (“Clinical outcomes of xeno-free expansion and transplantation of autologous ocular surface epithelial stem cells via contact lens delivery: a prospective case series” in Stem Cell Res. & Therapy (2015), 6(23), 1-14; DOI 10.1186/s13287-015-0009-1), the entire disclosure of which is hereby incorporated by reference. In some embodiments, exogenous MG53 is added to the dosage forms, or stem cells expressing MG53 are added to the dosage forms, or viral vectors that cause cells to express MG53 are added to the dosage forms, or embodiments of two or more such systems are employed in said dosage form(s).

Accordingly, the invention provides a coated contact lens dosage form comprising exogenously added MG53. The invention also provides a coated contact lens dosage form comprising cells that express MG53. The invention also provides a coated contact lens dosage form comprising a viral vector that causes cells to express MG53.

Compositions and dosage forms of the invention can further comprise one or more pharmaceutically acceptable excipients. Ophthalmic dosage forms can comprise one or more excipients independently selected at each occurrence from the group consisting of acidic agent, alkaline agent, buffer, tonicity modifier, osmotic agent, water soluble polymer, water-swellable polymer, thickening agent, complexing agent, chelating agent, penetration enhancer. Suitable excipients include U.S.F.D.A. inactive ingredients approved for use in ophthalmic formulations (dosage forms), such as those listed in the U.S.F.D.A's “Inactive Ingredients Database (available on the following website: https://www.fda.gov/Drugs/InformationOnDrugs/ucm13978.htm; October 2018), the entire disclosure of which is hereby incorporated by reference. Suitable excipients are also disclosed by Prasad et al. (“Excipients Utilized for Ophthalmic Drug Delivery Systems” in Nano-Biomaterials for Ophthalmic Drug Delivery” (Springer International Publishing, Switzerland (2016), pp 555-582; ISBN (print) 978-3-319-29344-8 or ISBN (online) 978-3-319-29346-2; https://doi.org/10.1007/978-3-319-29346-2_24; the entire disclosure of which is hereby incorporated by reference).

As used herein, an acidic agent is a compound or combination of compounds that comprises an acidic moiety. Exemplary acidic agents include organic acid, inorganic acid, mineral acid and a combination thereof. Exemplary acids include hydrochloric acid, hydrobromic acid, sulfuric acid, sulfonic acid, sulfamic acid, phosphoric acid, or nitric acid or others known to those of ordinary skill; and the salts prepared from organic acids such as amino acids, acetic acid, propionic acid, succinic acid, glycolic acid, stearic acid, lactic acid, malic acid, tartaric acid, citric acid, ascorbic acid, pamoic acid, maleic acid, hydroxymaleic acid, phenylacetic acid, glutamic acid, benzoic acid, salicylic acid, sulfanilic acid, 2-acetoxybenzoic acid, fumaric acid, toluenesulfonic acid, methanesulfonic acid, ethane disulfonic acid, oxalic acid, isethionic acid, others acids known to those of ordinary skill in the art, or combinations thereof.

As used herein, an alkaline agent is a compound or combination of compounds that comprises an alkaline moiety. Exemplary alkaline agents include primary amine, secondary amine, tertiary amine, quaternary amine, hydroxide, alkoxide, and a combination thereof. Exemplary alkaline agents include ammonia solution, ammonium carbonate, diethanolamine, monoethanolamine, potassium hydroxide, sodium borate, sodium carbonate, sodium bicarbonate, sodium hydroxide, triethanolamine, diethanolamine, monobasic phosphate salt, dibasic phosphate salt, organic amine base, alkaline amino acids and trolamine, others known to those of ordinary skill in the art, or combinations thereof.

Exemplary excipients (inactive ingredients as defined by the U.S.F.D.A.) that can be included in dosage forms of the invention include, by way of example and without limitation, water, benzalkonium chloride, glycerin, sodium hydroxide, hydrochloric acid, boric acid, hydroxyalkylphosphonate, sodium alginate, sodium borate, edetate disodium, propylene glycol, polysorbate 80, citrate, sodium chloride, polyvinylalcohol, povidone, copovidone, carboxymethylcellulose sodium, Dextrose, Dibasic Sodium Phosphate, Monobasic Sodium Phosphate, Potassium Chloride, Sodium Bicarbonate, Sodium Citrate, Calcium Chloride, Magnesium Chloride, stabilized oxychloro complex, Calcium Chloride Dihydrate, Erythritol, Levocarnitine, Magnesium Chloride Hexahydrate, Sodium Borate Decahydrate, Sodium Citrate Dihydrate, Sodium Lactate, Sodium Phosphate (Mono- and Dibasic-), Polyethylene Glycol 400, Hydroxypropyl Guar, Polyquaternium-1, Zinc Chloride, white petrolatum, mineral oil, hyaluronic acid, artificial tear, or combinations thereof.

It should be understood, that compounds used in the art of pharmaceutical formulations generally serve a variety of functions or purposes. Thus, if a compound named herein is mentioned only once or is used to define more than one term herein, its purpose or function should not be construed as being limited solely to that named purpose(s) or function(s).

As used herein, “pharmaceutically acceptable salts” refer to derivatives of the disclosed compounds wherein the compound is modified by making an acid or base salt thereof. Examples of pharmaceutically acceptable salts include, but are not limited to, mineral or organic acid salts of basic residues such as amines; alkali or organic salts of acidic residues such as carboxylic acids; and others known to those of ordinary skill. The pharmaceutically acceptable salts can be synthesized from the parent therapeutic compound which contains a basic or acidic moiety by conventional chemical methods. Lists of suitable salts are found in Remington's Pharmaceutical Sciences, 17th ed., Mack Publishing Company, Easton, Pa., 1985, p. 1418, the disclosure of which is hereby incorporated by reference.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

MG53 can be used in cotherapy or adjunctive therapy with one or more other active ingredients to treat ophthalmic diseases, disorders or conditions. Exemplary suitable active ingredients include, among others, U.S.F.D.A. approved drugs for ophthalmologic dosage forms. Such active ingredients include, by way of example and without limitation, the following. Even though specific diseases, disorders and conditions are listed for specific combinations, the invention includes other uses wherein said combinations are known or found to be therapeutically effective.
Luxturna (voretigene.neparvovec); Spark Therapeutics; for example, for the treatment of vision loss due to confirmed biallelic RPE65-mediated inherited retinal disease;
Rhopressa (netarsudil ophthalmic solution); Aerie Pharmaceuticals; for example, for the treatment of glaucoma or ocular hypertension;
Vvzulta (latanoprostene bunod ophthalmic solution); Bausch & Lomb; for example, for the reduction of intraocular pressure in patients with open-angle glaucoma or ocular hypertension;
Zerviate (cetirizine ophthalmic solution (0.24%); NicOx; for example, for the treatment of ocular itching associated with allergic conjunctivitis;
Humira (adalimumab); Abbvie; for example, for the treatment of uveitis;
Xiidra (lifitegrast); Shire; for example, for the treatment of dry eye disease;
Hetlioz (tasimelteon); Vanda Pharmaceuticals; for example, for the treatment of non-24-hour sleep-wake disorder in the totally blind;
Omidria (phenylephrine and ketorolac injection); Omeros; for example, for use during eye surgery to prevent intraoperative miosis and reduce post-operative pain;
Oralair (Sweet Vernal, Orchard, Perennial Rye, Timothy and Kentucky Blue Grass Mixed Pollens Allergen Extract); Greer Labs; for example, for the treatment of grass pollen-induced allergic rhinitis with or without conjunctivitis;
Cystaran (cysteamine hydrochloride); Sigma Tau Pharmaceuticals; for example, for the treatment of corneal cystine crystal accumulation due to cystinosis;
Jetrea (ocriplasmin; Thrombogenics; for example, for the treatment of symptomatic vitreomacular adhesion;
Lucentis (ranibizumab injection); Genentech; for example, for the treatment of diabetic macular edema;
Zioptan (tafluprost ophthalmic solution); Merck; for example, for the treatment of elevated intraocular pressure;
Evlea (aflibercept); Regeneron Pharmaceuticals; for example, for the treatment of neovascular (wet) age-related macular degeneration;
Zyrnaxid (gatifloxacn ophthalmic solution); Allergan; for example, for the treatment of bacterial conjunctivitis;
Acuvail (ketorolac tromethamine); Allergan; for example, for the treatment of pain and inflammation following cataract surgery;
Bepreve (bepotastine besilate ophthalmic solution); Ista Pharmaceuticals; for example, for the treatment of itching associated with allergic conjunctivitis;
Besivance (besifloxacin ophthalmic suspension); Bausch & Lomb; for example, for the treatment of bacterial conjunctivitis;
Ozurdex (dexamethasone); Allergan; for example, for the treatment of macular edema following branch retinal vein occlusion or central retinal vein occlusion;
Zirgan (ganciclovir ophthalmic gel); Sirion Therapeutics; for example, for the treatment of acute herpetic keratitis;
Akten (lidocaine hydrochloride); Akom; for example, for anesthesia during ophthalmologic procedures;
Astepro (azelastine hydrochloride nasal spray); Meda Pharmaceuticals Inc; for example, for the treatment of seasonal and perennial allergic rhinitis;
DureZol (difluprednate); Sirion Therapeutics; for example, for the treatment of inflammation and pain associated with ocular surgery;
AzaSite (azithromycin); InSite Vision; for example, for the treatment of bacterial conjunctivitis;
Lucentis (ranibizumab); Genentech; for example, for the treatment of neovascular (wet) age related macular degeneration;
Macugen (pegaptanib); Pfizer/Eyetech Pharmaceuticals; for example, for the treatment of wet age-related macular degeneration;
Restasis (cyclosporine ophthalmic emulsion); Allergan; for example, for the treatment of low tear production;
Lumigan (bimatoprost ophthalmic solution); Allergan; for example, for the reduction of intraocular pressure in patients with open-angle glaucoma or ocular hypertension;
Travatan (travoprost ophthalmic solution); Alcon; for example, for the reduction of elevated intraocular pressure in patients with open-angle glaucoma or ocular hypertension;
Valcyte (valganciclovir HCl); Roche; for example, for the treatment of cytomegalovirus retinitis in patients with AIDS;
Betaoxn (levobetaxolol hydrochloride suspension, drops); Alcon; for example, for lowering IOP in patients with chronic open-angle glaucoma or ocular hypertension;
Quixin (levofloxacin); Santen; for example, for treatment of bacterial conjunctivitis;
Rescula (unoprostone isopropyl ophthalmic solution) 0.15%; Ciba Vision; for example, for the treatment of open-angle glaucoma or ocular hypertension;
Visudyne (verteporfin for injection): QLT; for example, for the treatment of wet age-related macular degeneration (wet AMD);
Alamast (Pemirolast potassium ophthalmic solution); Santen; pemirolast potassium ophthalmic solution;
ZADITOR (ketotifen fumarate ophthalmic solution; 0.025%); Ciba Vision; for example, for the prevention of itching of the eye;
Alrex; Bausch & Lomb, Pharmos; for example, for the treatment of seasonal allergic conjunctivitis;
Cosopt (Trusopt (dorzolamide) and Timoptic (timolol)); Merck; for example, for the treatment of glaucoma or ocular hypertension;
Lotemax (loteprednol etabonate; site-specific corticosteroid); Bausch & Lomb, Pharmos; for example, for the treatment of post-operative eye inflammation;
Salagen (pilocarpine HCl); MGI Pharma; for example, for the treatment of Sjogren's Syndrome;
Viroptic (trifluridine 1%); King Pharmaceuticals; for example, for the treatment of inflammation of the cornea in children due to herpes simplex virus;
Vitravene Injection (fomivisen); Isis Pharmaceuticals; for example, for the treatment of CMV in AIDS patients;
Acluar (ketorolac tromethamine ophthalmic solution) 0.5; Allergan; for example, for the treatment of postoperative inflammation in patients who have undergone cataract extraction;
Acular (ketorolac tromethamine ophthalmic solution) 0.5%; Allergan; for example, for the treatment of post-surgical inflammation following cataract extraction;
BSS Sterile Irrigating Solution; Alcon; for example, for treatment during ocular surgical procedures;
AK-Con-A (naphazoline ophthalmic); Akom; Over-the-counter combination vasoconstrictor/antihistamine product for ophthalmic use;
Alphagan (brimonidine); Allergan; for example, for the treatment of open-angle glaucoma and ocular hypertension;
Ocuflox (ofloxacin ophthalmic solution) 0.3%; Allergan; for example, for the treatment of corneal ulcers;
OcuHist (pheniramine maleate; 0.3%); Pfizer; Over-the-counter antihistamine eye drop;
Vistide (cidofovir); Gilead; for example, for the treatment of cytomegalovirus (CMV) retinitis; and/or
Vitrasert Implant (ganciclovir); Chiron; Drug delivery system for the treatment of cytomegalovirus.

Other active ingredients that can be used in cotherapy or adjunctive therapy with MG53 include, by way of example and without limitation, ketotifen funmarate, naphazoline hydrochloride, Allium cepa 6×; Apis 6; Sabadilla 6×; Euphrasia (Eyebright) 4×, Cineraria maritima 5×; Causticum 8×; Cal. phos. 11×; Euphrasia 6×; Sepia 6×; Silicea 11×; Calc. flour 11×, tetrahydrozoline hydrochloride, ciprofloxacin, levofloxacin, moxifloxacin, tobramycin, doxycycline, NSAID (non-steroidal anti-inflammatory drug), steroid, corticosteroid, antihistamine, mast cell stabilizer, cyclosporine, latanoprost, Neomycin Sulfate (equivalent to 3.5 mg neomycin base), Polymyxin B Sulfate equivalent to 10,000 polymyxin B units, and Bacitracin Zinc equivalent to 400 bacitracin units, calcineurin inhibitor, erythromycin, cephalosporin, integrin antagonist, autologous blood serum, antiviral drug, fomivirsen, corticosteroid, loteprednol, loteprednol etabonate, carbonic anhydrase inhibitor, anti-glaucoma agent, non-selective beta blocker, tumor necrosis factor (TNF) blocker, tetracyclic antibiotic, aminoglycoside, or combinations thereof.

The therapeutically acceptable dose, maximum tolerated dose (MTD), and minimally effective dose (MED) for each of said active ingredients is well known and set forth in the respective U.S.F.D.A. approved product package insert for each said active ingredients.

A composition, dosage form or formulation of the invention can include one, two or more active ingredients in combination with MG53. The dose of each said active ingredient in said composition, dosage form or formulation of the invention will be a therapeutically effective dose including and above the MED and including and below the MTD.

In some embodiments, the combination treatment of MG53 with another active ingredient provides at least additive therapeutic efficacy. In some embodiments, said combination provides synergistic therapeutic efficacy. In some embodiments, MG53 reduces the occurrence of, reduces the level of, or eliminates adverse events caused by the other active ingredient. In some embodiments, MG53 repairs injury caused by the other active ingredient.

Diseases, disorders or conditions that can be treated with the MG53-containing composition, dosage form or formulation of the invention (with or without additional active ingredient(s) as may be required or clinically indicated) include but are not limited to: vision loss due to confirmed biallelic RPE65-mediated inherited retinal disease, glaucoma or ocular hypertension, intraocular pressure in patients with open-angle glaucoma or ocular hypertension, ocular itching associated with allergic conjunctivitis, uveitis, dry eye disease, non-24-hour sleep-wake disorder in the totally blind, during eye surgery to prevent intraoperative miosis and reduce post-operative pain, grass pollen-induced allergic rhinitis with or without conjunctivitis, corneal cystine crystal accumulation due to cystinosis, symptomatic vitreomacular adhesion, diabetic macular edema, elevated intraocular pressure, neovascular (wet) age-related macular degeneration, pain and inflammation following cataract surgery, itching associated with allergic conjunctivitis, bacterial conjunctivitis, macular edema following branch retinal vein occlusion or central retinal vein occlusion, acute herpetic keratitis, anesthesia during ophthalmologic procedures, seasonal and perennial allergic rhinitis, inflammation and pain associated with ocular surgery, wet age-related macular degeneration, low tear production, cytomegalovirus retinitis in patients with AIDS, itching of the eye, post-operative eye inflammation, Sjogren's Syndrome, inflammation of the cornea in children due to herpes simplex virus, preoperative and postoperative ocular surgical procedures, corneal ulcers, meibomian gland dysfunction, keratoconjunctivitis, Autoimmune Keratoconjunctivitis Sicca.

The acceptable concentrations of said excipients are well known in the art and specific concentrations (amounts) thereof are set forth in the package insert or package label of known commercial products containing the same.

The ophthalmic dosage form or composition is preferably isotonic or approximately (about) isotonic. In some embodiments, the ophthalmic dosage form comprises about 0.7-1.1 wt % or about 0.8-1.0 wt % or about 0.9 wt % of osmotic salt, such as NaCl. In some embodiments, the ophthalmic dosage form preferably has a pH in the range of about 6.5-7.6, with a mean of about pH 7.

It should be understood, that compounds used in the art of pharmaceutics may serve a variety of functions or purposes. Thus, if a compound named herein is mentioned only once or is used to define more than one term herein, its purpose or function should not be construed as being limited solely to that named purpose(s) or function(s).

In the examples below, ranges are specified for the amount of each ingredient. Ranges including “0” as the lowest value indicate an optional ingredient. The lower limit “>0” indicates the respective material is present.

As used herein, the terms “about” or “approximately” are taken to mean a variation or standard deviation of 10%, ±5%, or ±1% of a specified value. For example, about 20 mg is taken to mean 20 mg±10%, which is equivalent to 18-22 mg.

As used herein, the term “prodrug” is taken to mean a compound that, after administration, is converted within a subject's body, e.g. by metabolism, hydrolysis, or biodegradation, into a pharmacologically active drug. The prodrug may be pharmacologically active or inactive. For example, a prodrug of MG53 (native or mutant) would be converted to the native form or mutant form, respectively, of MG53. The term “precursor” may also be used instead of the term “prodrug”.

As used herein, the term “derivative” is taken to mean: a) a chemical substance that is related structurally to a first chemical substance and theoretically derivable from it; b) a compound that is formed from a similar first compound or a compound that can be imagined to arise from another first compound, if one atom of the first compound is replaced with another atom or group of atoms; c) a compound derived or obtained from a parent compound and containing essential elements of the parent compound; or d) a chemical compound that may be produced from first compound of similar structure in one or more steps. For example, a derivative may include a deuterated form, oxidized form, dehydrated, unsaturated, polymer conjugated or glycosilated form thereof or may include an ester, amide, lactone, homolog, ether, thioether, cyano, amino, alkylamino, sulfhydryl, heterocyclic, heterocyclic ring-fused, polymerized, pegylated, benzylidenyl, triazolyl, piperazinyl or deuterated form thereof.

In the examples below, ranges are specified for the amount of each ingredient. Ranges including “0” as the lowest value indicate an optional ingredient. Compositions with quantities of ingredients falling within the compositional ranges specified herein were made. Compositions of the invention comprising quantities of ingredients falling within the compositional ranges specified herein operate as intended and as claimed.

In view of the above description and the examples below, one of ordinary skill in the art will be able to practice the invention as claimed without undue experimentation. The foregoing will be better understood with reference to the following examples that detail certain procedures for the preparation and use of compositions according to the present invention. All references made to these examples are for the purposes of illustration. The following examples should not be considered exhaustive, but merely illustrative of only a few of the many embodiments contemplated by the present invention. The methods described herein can be followed to prepare and use compositions of the invention and to practice methods of the invention.

### Example 1

### rhMG53 Protein Production and Quality Control

The following process was used to produce native MG53 protein.

E. coli fermentation was used to obtain high quality (>97% purity) rhMG53 (recombinant human MG53) protein as described by Zhu et al. (“Polymerase transcriptase release factor (PTRF) anchors MG53 protein to cell injury site for initiation of membrane repair” in The Journal of biological chemistry (2011), 286, 12820-12824) and Weisleder et al. (Recombinant MG53 protein modulates therapeutic cell membrane repair in treatment of muscular dystrophy. Science translational medicine (2012), 4, 139ra185), the entire disclosures of which are hereby incorporated by reference. The membrane protective activity of rhMG53 from each preparation was determined with established micro-glass bead injury assay as described previously (ibid).

### Example 2

### Corneal Fibroblasts Cell Culture

Human telomerase-immortalized corneal epithelial cells (hCEC; generously provided by Dr. Danielle Robertson, University of Texas Southwestern) were maintained in keratinocyte growth medium (KGM)-2 supplemented with KGM-2 SingleQuot Kit Supplements and Growth Factors (Lonza, Basel, Switzerland), in a 5% CO₂ humidified incubator at 37° C., and passaged every 3 to 5 days.

Primary corneal fibroblasts were prepared from superficial keratectomy samples obtained from the axial cornea of cadaveric canine globes. First, epithelium was mechanically debrided and explants, approximately 5 mm in diameter and 250 μm in depth, comprised of stromal tissue only were place in culture dishes containing maintained in Dulbecco's modified Eagle's medium (DMEM) supplemented with 10% fetal bovine serum and 1% penicillin/streptomycin in a 5% CO₂ incubator. Western blot analysis evaluating vimentin and cytokeratin expression verified the stromal origin of the cells.

For treatment with TGF-β and rhMG53, fibroblasts (seeded at 5×10⁴ cells/cm²) grew to 70% confluence, before being washed twice with serum-free media and subjected to treatment in serum-free DMEM. Cells were treated with DMEM (control), in the presence of either TGF-β (10 ng/mL), rhMG53 (50 μg/mL), or a combination of both TGF-β and rhMG53 for varying times to investigate myofibroblast differentiation.

### Example 3

### Cell Scratch Injury Healing Assay

An in vitro scratch test was performed using in serum-starved cells. To generate myofibroblasts, fibroblasts were first treated with TGF-β (10 ng/mL). Cells were allowed to grow to 90% confluence and a 1-mm scratch was subsequently made in the cellular monolayer before being treated with 0 or 50 μg/mL rhMG53. Photomicrographs were taken immediately after the scratch and then every 8 hours until restoration of the monolayer. ImageJ software (National Institutes of Health, Bethesda, Md.) was used to quantify the change in area over time.

### Example 4

### Confocal Live Cell Imaging and Conjugation of Alexa647 to Cells and MG53

For live cell imaging of MG53-mediated cell membrane repair in corneal epithelial cells, transfection of GFP-MG53 into hCECs was performed using the Lipofectamine LTX reagent (Life Technologies), per manufacturer's instructions. hCECs expressing GFP-MG53 were subsequently subjected to microelectrode penetration-induced injury to the plasma membrane as previously described¹². Cells were imaged using confocal microscopy (Zeiss LSM780). For visualizing the dynamic process of rhMG53 entry into the corneal fibroblasts, rhMG53 and bovine serum albumin (BSA) were labeled with Alexa Fluor™ 647 by Alexa Fluor™ 647 Protein Labeling Kit (Life Technologies, Cat. No. A20173). Labeled rhMG53 or BSA was added to the culture medium of primary corneal fibroblasts and intracellular signal of Alexa 647 was imaged at indicated time points by a confocal microscope. Intracellular fluorescent intensity of Alexa 647 at each time point was quantified by ImageJ software. To visualize cell morphology for fluorescence quantification, the cells were counterstained with MitoTracker Green (Life Technologies, Cat. No. M7514).

### Example 5

### CRISPR/Cas9 Mediated MG53 Knockout

CRISPR/Cas9 mediated MG53 knockout was performed following the methods described by Ji Y M. et al. (“DEPTOR suppresses the progression of esophageal squamous cell carcinoma and predicts poor prognosis” in Oncotarget (2016), 7, 14188-14198) and Xu L et al. (“CRISPR-mediated Genome Editing Restores Dystrophin Expression and Function in mdx Mice” in Molecular therapy: the journal of the American Society of Gene Therapy (2016), 24, 564-569), the entire disclosures of which are hereby incorporated by reference.

Briefly, 2×10⁵ hCEC cultured in antibiotic-free medium were plated in 6-well plates. The guide RNA probe sequences were obtained from CRISPR design (http://crispr.mit.edu/). Total two guide RNA sequences (5′-AGAACGGTGCCATCCGCCGC-3′ (SEQ ID NO. 1) and 5′-CGGGCGCGTCGAACAGCTGC-3′ (SEQ ID NO. 2) were tested and the one (5′-AGAACGGTGCCATCCGCCGC-3′ (SEQ ID NO. 1) with higher knockout efficiency was used in our experiments. Twenty-four hours later, after cells reached 80% confluence, CRISPR/Cas9 MG53 plasmid was transfected into the hCEC using Lipofectamine 3000, according to the manufacturer's instructions. Forty-eight hours post-transfection, the culture medium was aspirated and replaced with fresh medium containing puromycin (1 μg/mL) to select and establish the stably transfected cells.

### Example 6

### In Vitro Cell Membrane Injury Assay

In vitro cell membrane injury repair assay was performed as described by Zhu et al. (“Polymerase transcriptase release factor (PTRF) anchors MG53 protein to cell injury site for initiation of membrane repair” in The Journal of biological chemistry (2011), 286, 12820-12824) and Weisleder et al. (Recombinant MG53 protein modulates therapeutic cell membrane repair in treatment of muscular dystrophy. Science translational medicine (2012), 4, 139ra185), the entire disclosures of which are hereby incorporated by reference.

hCECs were suspended in Dulbecco's PBS at a concentration of 6.0×10⁵ cells/mL; 150 μL of this cell suspension (9×10⁴ hCECs) was added to each well of a 96-well plate with acid-washed glass micro-beads and the indicated dose of rhMG53 (0-200 μg/mL). To induce cell membrane damage, the plate was shaken at 200 rpm for six minutes. Plates were then centrifuged at 3000×g for five minutes and 50 μL supernatant was removed. Lactate dehydrogenase (LDH) activity of the supernatant was determined using a LDH Cytotoxicity Detection Kit (TaKaRa). The LDH values from wells without glass beads (no damage) were used to determine the background activity for each condition and were subtracted from experimental values before comparison.

### Example 7

### Western Blot

Protein lysates from indicated tissue and cell sources were separated by SDS-PAGE. Proteins were transferred from gels to PVDF membranes at 4° C. The blots were washed with PBST (PBS+0.5% Tween-20), blocked with 5% milk in PBST for 2 hours, and incubated with indicated primary antibodies overnight at 4° C. under rotation. Secondary antibodies, anti-mouse or anti-rabbit IgG HRP conjugated, were applied at 1:5000 dilution and incubated for approximately 1.5 hours with shaking at room temperature. Immunoblots were visualized with an ECL plus kit (Pierce). The antibodies used in this study were as follows: rabbit anti-MG53 antibody was generated by our laboratory and the sensitivity and specificity were previously confirmed: anti-p-Smad2 antibody (Cell Signaling Technology, Cat. No. 3108); anti-Smad2 antibody (Cell Signaling Technology, Cat. No. 5339); anti-Smad5, (Cell Signaling Technology, Cat. No. 12534); anti-p-Smad5 antibody (Cell Signaling Technology, Cat. No. 9516); anti-GAPDH antibody (Cell Signaling Technology, Cat. No. 2118s); anti-alpha-SMA antibody (Invitrogen, Cat. No. 14-9760-82); and anti-fibronectin antibody (Sigma-Aldrich, Cat. No. F3648).

Semi-quantitative analysis was performed to quantify expression levels of MG53 in canine aqueous humor and human tear samples. Briefly, 0.4 ng purified rhMG53 protein and 20 μL aqueous humor or tear samples were subjected to Western blot analysis. Western blot bands were quantified using ImageJ software (NIH) and the concentration of MG53 in samples were calculated based on signal ratio between purified rhMG53 protein and average of aqueous humor and tear samples.

### Example 8

### Quantitative RT-PCR

The expression pattern of α-SMA and fibronectin in treated or untreated canine corneal fibroblasts were examined by quantitative real-time PCR (qRT-PCR) analysis. Total RNAs were extracted by using TRIzol reagent (Invitrogen, CA, Cat. No. 15596026), and genome DNA contamination was eliminated by DNase I (Invitrogen, CA, Cat. No. 18047019), according to the manufacturer's instructions. One microgram of total RNA was reverse transcribed by cDNA synthesis (Thermo Scientific, Cat. No. 1651) and the products were subjected to quantitative real-time PCR, carried out by SYBR Green Real-Time PCR Mix (Thermo Scientific, Cat. No. A25778) on the DNA Engine LightCycler 480 Instrument II (Roche Molecular Systems, Inc,). The canine gene GAPDH was used as an internal control. The canine primers used in the assay were: α-SMA forward: 5′-AACACGGCATCATCACCAA-3′ (SEQ ID NO. 3), α-SMA reverse: 5′-AGGCGTAGAGGGAAAGCA-3′ (SEQ ID NO. 4); fibronectin forward: 5′-CCTCTGACGGCGGAACAAACGACCA-3′ (SEQ ID NO. 5), fibronectin reverse: 5′-AGAGGGTCCCACGTTGTACTGCTTG-3′ (SEQ ID NO. 6), GAPDH forward: 5′-GTGAAGGTGGAGTGAACGGAITG-3′ (SEQ ID NO. 7), GAPDH reverse: 5′-TTGATGTTGGCGGGAT-3′ (SEQ ID NO. 8).

### Example 9

### In Vivo Corneal Alkaline Injury Healing Models

All animal care and usage followed NIH guidelines and were in accordance with the ARVO Statement for the Use of Animals in Ophthalmic and Vision Research. Rodent studies received IACUC approval by The Ohio State University. For all corneal injury healing models, injury was induced under anesthesia and all animals received topical antibiotics, and topical and systemic analgesics for at least 72 hours for pain management.

To evaluate presence of MG53 in the mediums surrounding the cornea, aqueous humor was collected from normal canine cadaveric globes immediately following enucleation. All samples were immediately frozen at −80° C. until analysis.

mg53−/− mice and their wild type littermates were generated, bred and genotyped as previously described¹². To ensure data reproducibility, mouse tail samples are retained and cataloged for secondary future validation, if necessary. The mg53−/− mouse line has been previously validated. A 2-mm filter paper disc soaked in 1N NaOH was applied to the axial cornea to induce injury. The clinical opacity and vascularization scores were determined by a masked, board certified veterinary ophthalmologist (AGM), using a modified Hackett-McDonald scoring system. Fourteen days post-alkaline injury, the mice were sacrificed and eyes underwent analyses. In order to obtain details regarding the injury response in mg53−/− and wt corneas, globes were fixed either for horizontal sectioning or flat mount staining. Analysis of all tissues was performed by an individual masked to the genotype.

For rhMG53 treatment of corneal injury, Wistar rats (Harlan Laboratories; Indianapolis, Ind.) were used and ophthalmic exams were performed by a masked board-certified veterinary ophthalmologist. An injury was introduced by placing a 3-mm piece of filter paper soaked in 1N NaOH on the axial cornea, thus removing the anterior stroma and overlaying epithelium. Injured corneas received topical sterile saline with 0 or 100 ng/mL rhMG53, twice daily for a total of seven days. Size and depth of the corneal injury was verified daily using fluorescein. In conjunction with the use of fluorescein dye to monitor healing rates, the following were clinically evaluated: corneal opacification, vascularization, and cellular infiltrate, conjunctival edema, hyperemia, and discharge, and presence of uveitis. At 7 days following treatment, animals were euthanized, and globes were enucleated. In all subsequent histologic analyses, the individual was masked to the treatment.

### Example 10

### Human Subjects for Tear Collection

A total of 6 volunteers were enrolled and only adults 18 years and older were included. Before initiation of the study, this research was approved by The Ohio State University Institutional Review Board. Participants signed an informed consent document following the tenets of the Declaration of Helsinki. Non-reflex tears were collected from the inferior tear prism using 5 μL Drummond glass microcapillary tubes. All samples were immediately frozen at −80° C. until analysis.

### Example 11

### Histopathology and Immunohistochemistry

Enucleated globes from mice or rats were fixed in 10% formalin overnight at 4° C. After fixation, samples were washed three times for 5 min with 70% ethanol. Washed samples were processed and embedded in paraffin. Four μm thick paraffin sections were cut and stained with Hematoxylin-Eosin (H&E). Under light microscopy, the number of epithelial cell layers present was manually counted in four areas within the axial cornea.

Immunofluorescent staining of MG53 was performed using flat mount corneas following a previously published study-. Briefly, enucleated globes were fixed in 4% PFA for 1 hr at 4° C. Corneas were carefully dissected from the eye (Leica, Stereo Zoom 4 Microscope) and returned to 4% PFA for fixation overnight at 4° C. Subsequently, corneas were incubated in blocking buffer (0.5% Triton X-100 and 5% goat serum in PBS) for at least 2 hr at room temperature to permeabilize the tissue and prevent nonspecific binding of the primary antibody. Anti-CD31 (BD Biosciences, Cat. No. 550274); and anti-αSMA (Invitrogen, Cat. No. 14-9760-82) antibodies in blocking buffer was applied to the tissue and incubated at 4° C. overnight. After six washes (1 hour per wash at room temperature) with washing buffer (0.5% Triton X-100 in PBS), a secondary antibody in blocking buffer was applied to the cornea and incubated overnight at 4° C. Tissues were washed three times with PBS for 1 hour each at room temperature. Corneas were transferred into fresh PBS and four incisions from periphery towards the center were made to facilitate imaging.

### Example 12

### Methods for Cotherapy or Adjunctive Therapy

The following cotherapeutic or adjunctive therapeutic methods of treatment are used to treat diseases, disorders or conditions that are therapeutically responsive to MG53. The classes of active ingredients specified in combination with MG53 are administered to subjects in need thereof.

### Ophthalmic Bacterial Infection

An ophthalmic bacterial infection is treated by administration of MG53 with an antibiotic (antibacterial). The MG53 and antibiotic are administered in the same or different dosage forms and are administered at the same time, at overlapping times, or at spaced-apart times.

The dosage form is independently selected at each occurrence from the group consisting of liquid solution, gel, cream, ointment, collagen shield, slab gel, implant, amniotic membrane, autologous blood serum, and coated contact lens.

### Example 13

### Preparation of Plasmid for Antibiotic Inducible Secretion MG53

The following process was used to make Tet-tPA-MG53 plasmid.

The plasmid was made by adding a tissue plasminogen activator (tPA) leader sequence (tPA-MG53 sequence) ahead of the human mg53 cDNA. The tPA-MG53 sequence was cloned behind a minimum CMV promoter that is under the control of a tetracycline response element (TRE). Although optional, this plasmid also contained a SV40-driven transcription of mCherry fluorescent marker, allowing for visualization and selection of transfected cells. The resulting plasmid is generally described in FIGS. 10 and 11.

### Example 14

### Packaging of Plasmid into Adenovirus and Infection of Limbal Stem Cells

The following process was used to make adenovirus vector with antibiotic inducible gene expression of MG53. The plasmid of Example 13 was cloned into the pShuttle vector to generate the pShuttle-Tet-ON-tPA-MG53, which was then electroporated into AdEz competent cells. This allows for generation of AdEz that encapsulate the pShuttle-Tet-ON-tPA-MG53 plasmid. The recombinant AdEz-plasmids were finally transfected into 293 AD cells via lipofection. After 2-4 weeks, adenovirus packaged with Tet-ON-tPA-MG53 was collected from the cell culture medium.

Limbal stem cells were then infected with the Tet-tPA-MG53 adenoviral vector. Corneal limbus stem cells were seeded on 6 wells plate until 50-60% confluent. Then the cells were infected with tet-on tPA-MG53 adenovirus (10 ul adenovirus diluted in 2 ml culture medium). After overnight infection, the medium was changed. After further incubated for 48 h, 1 μg/mL of Doxycycline was added, and after 24 h of DOX induction, the medium and cell were harvest for western blotting.

This procedure was used to prepare adenovirus vectors having the plasmid maps depicted in FIGS. 11 and 12.

### Example 15

### Isolation and Culture of LSC's from Mouse

Mouse eyeballs were dissected out after sacrificing the mice via cervical dislocation. Limbal regions were carefully dissected out under a dissecting microscope and washed in cold PBS with 1% penicillin and streptomycin, and then cut into small pieces. Cell clusters were obtained by 0.2% collagenase IV digestion at 37° C. for 1 h, single cells were obtained by further digestion with 0.025% trypsin-EDTA at 37° C. for 20 min. Centrifuge the cells for 5 min at 350×g, and plate in culture dish.

The culture medium was comprised the following: DMEM/F12 basal medium (3:1) with 1% penicillin-streptomycin, 10% fetal bovine serum, 1% antibiotic-antimycotic, 10 ng/ml EGF, 5 μg/ml insulin, 0.1 nM cholera toxin.

For proof of identity of the mouse LSC's, the LSCs were plated on the glass bottom dishes. When they reached confluency of 70-80%, the cells were fixed with 4% paraformaldehyde and permeablized with 1% Triton X100. The cells then were stained with antibody against ΔNp63α, a limbal stem cells marker. The stained cells were examined on confocal microscope to make sure 90% of cells are positive for ΔNp63α.

### Example 16

### Autologous Blood Serum Dosage Form

### Preparation of Autologous Serum

Blood is collected from a subject After about 0-48 h at 21 C, the blood is centrifuged for about 5-20 min at 300-4000 g force. The supernatant serum is collected and then diluted 20-100% with BSS (balanced salt solution; about 0.9% wt NaCl in water) optionally containing chloramphenicol o other antibiotic or preservative. Exogenous rhMG53 (10-500 ng/ml; 50-250 ng/ml, or about 100 ng/ml) is added to the autologous serum. The serum can be applied one to ten times, one to five times or one to three times daily or any other dosing frequency determined to be therapeutically effective.

### Example 17

### Amniotic Membrane Dosage Form

Amniotic membrane (commercially available, such as Amiodisk, etc) is soaked in artificial tear containing rhMG53 protein before applying to ocular surface.

### Example 18

### Collagen Shield Dosage Form

In general, collagen shields are manufactured from porcine scleral tissue or bovine corium (dermis) collagen and contain mainly type I collagen and some type III collagen. They are shaped like a contact lens and are supplied in a dehydrated form, requiring rehydration prior to insertion. Variations in collagen crosslinking can be induced with ultraviolet light (UV) during manufacture dictate lens duration before dissolution. Three different collagen shields are currently available with dissolution times of 12, 24, and 72 hours. Corneal collagen shields have a diameter of 14.5-16.0 mm, a base curve of 9 mm, and a central thickness of 0.15-0.19 mm. A study of the oxygen transmissibility of Bio-Cor (Bausch and Lomb, Clearwater, Fla.) collagen shields in vitro indicated that they behave like a 63% water-content hydrogel contact lens with an average oxygen permeability (Dk/L) of 27 1011 cm² mL 02/s mL mm Hg. Water-soluble compounds are trapped within the collagen matrix and some drugs undergo reversible binding to collagen. The collagen shield becomes saturated when placed in an aqueous solution comprising MG53; greater drug absorption occurs when the shield is soaked with higher protein concentrations. Therefore, the collagen shields can be soaked in saline solution containing rhMG53 protein (100 ng/ml).

### Example 19

### VV-MG53-LSC Dosage Form

The viral vector infected LSC's of Example 14 are placed in a carrier comprising two or more pharmaceutically acceptable excipients, and administered to a subject in need thereof. The carrier can be any carrier described herein or determined to be suitable for ophthalmic administration.

### Example 20

### Limbal Stem Cell-Coated Contact Lens

The following process was used to make a contact coated with bioengineered LSC's. The general procedure of Bobba et al. (“Clinical outcomes of xeno-free expansion and transplantation of autologous ocular surface epithelial stem cells via contact lens delivery: a prospective case series” in Stem Cell Res. & Therapy (2015), 6(23), 1-14; DOI 10.1186/s13287-015-0009-1), the entire disclosure of which is hereby incorporated, was followed except that the bioengineered limbal stem cells described herein were used to coat the contact lens.

The bioengineered stem cells are placed in autologous serum and a portion thereof is placed on the concave surface of a siloxane-hydrogel extended-wear CL (Lotrafilcon A; CIBA Vision, Duluth, Ga., USA) in 24-well culture plates (Corning Inc., Corning, N.Y., USA) in Eagle's minimum essential medium containing 10% autologous serum with antibiotic supplements. Cultures are kept in an isolated incubator set to 37° C. with 5% CO₂, and growth is monitored daily with media changed on alternate days. When cells reach confluence (9 to 16 days), patients are scheduled for the procedure and the cell-coated CL transported to the operating theatre in growth media in cold storage (4° C. to 10° C.). This ensure that cell activity can be preserved in the event of delays in theatres. The contact lens is then applied to the patient's injured cornea.

If needed, prior to insertion of the coated contact lens, 5% betadine is applied to the eye and a total superficial keratectomy, including removal of limbal epithelium is performed to remove any irregular epithelium or pannus or both. The contact LSC's is inserted onto the patient's ocular surface under topical anesthesia (Minims Benoxinate Hydrochloride 0.4%; Chauvin Pharmaceuticals, Bausch &Lomb. For prophylaxis against infection, each patient can be prescribed Minims Chloramphenicol 0.5% (Chauvin Pharmaceuticals, Bausch & Lomb), which is applied for 4 weeks. Patients may also receive Minims Dexamethasone sodium phosphate 0.1% (Chauvin Pharmaceuticals, Bausch &Lomb) tapered over the course of 1 month. Patients may also receive Minims Prednisolone sodium phosphate 0.5% (Chauvin Pharmaceuticals, Bausch & Lomb). The topical steroid regime is determined by the treating according to the degree of postoperative inflammation.

All data are expressed as mean±S.D. Groups were compared by Student's t test and analysis of variance for repeated measures. A value of p<0.05 was considered significant.

All values disclosed herein may have standard technical measure error (standard deviation) of ±10%. The term “about” or “approximately” is intended to mean±10%, ±5%, ±2.5% or ±1% relative to a specified value, i.e. “about” 20% means 20±2%, 20±1%, 20±0.5% or 20±0.25%. The term “majority” or “major portion” is intended to mean more than half, when used in the context of two portions, or more than one-third, when used in the context of three portions. The term “minority” or “minor portion” is intended to mean less than half, when used in the context of two portions, or less than one-third, when used in the context of three portions. It should be noted that, unless otherwise specified, values herein concerning pharmacokinetic or dissolution parameters are typically representative of the mean or median values obtained.

The above is a detailed description of particular embodiments of the invention. It will be appreciated that, although specific embodiments of the invention have been described herein for purposes of illustration, various modifications may be made without departing from the spirit and scope of the invention. Accordingly, the invention is not limited except as by the appended claims. All of the embodiments disclosed and claimed herein can be made and executed without undue experimentation in light of the present disclosure.

1) A method of treating corneal injury, the method comprising administering to the injured cornea of a subject an effective amount of MG53 in a dosage form.

2) (canceled)

3) (canceled)

4) The method of claim 1, wherein said dosage form a) releases or provides MG53 into or onto the eye; b) enables expression of MG53 followed by release of MG53 to the cornea or other eye tissue; c) comprises a stem cell comprising exogenously added MG53; d) comprises a limbal stem cell comprising exogenously added MG53; e) comprises an autologous serum dosage form comprising exogenously added MG53; f) comprises an autologous serum dosage form comprising cells that express MG53; g) comprises an autologous serum dosage form comprising a viral vector that causes cells to express MG53; h) comprises a collagen shield dosage form comprising exogenously added MG53; i) comprises a collagen shield dosage form comprising cells that express MG53; j) comprises a collagen shield dosage form comprising a viral vector that causes cells to express MG53; k) comprises an amniotic membrane dosage form comprising exogenously added MG53; l) comprises an amniotic membrane dosage form comprising cells that express MG53; m) comprises an amniotic membrane dosage form comprising a viral vector that causes cells to express MG53; n) comprises a coated contact lens dosage form comprising exogenously added MG53; o) comprises a coated contact lens dosage form comprising cells that express MG53; or p) comprises a coated contact lens dosage form comprising a viral vector that causes cells to express MG53.

5) (canceled)

6) (canceled)

7) The bioengineered stem cell of claim 8, wherein said bioengineered stem cell is a bioengineered limbal stem cell that expresses or comprises MG53.

8) A bioengineered stem cell that expresses or comprises MG53.

9) The bioengineered stem cell of claim 8, wherein said bioengineered stem cell comprises a) a viral vector comprising a plasmid that induces expression of MG53 in said bioengineered stem cell; b) a viral vector that causes said stem cell to express MG53; or c) Tet-tPA-MG53 plasmid.

10) (canceled)

11) (canceled)

12) (canceled)

13) (canceled)

14) (canceled)

15) (canceled)

16) (canceled)

17) (canceled)

18) (canceled)

19) (canceled)

20) (canceled)

21) (canceled)

22) (canceled)

23) (canceled)

24) (canceled)

25) (canceled)

26) (canceled)

27) (canceled)

28) (canceled)

29) (canceled)

30) (canceled)

31) A viral vector (VV) that induces expression of MG53 in a stem cell (SC).

32) The viral vector of claim 31, wherein said viral vector comprises a) a plasmid that induces expression of MG53 in stem cells following infection of said stem cells with said viral vector; b) adeno-associated virus (AAV) comprising a plasmid comprising a tissue plasminogen activator (tPA) leader sequence ahead of a human MG53 cDNA, thereby forming a tPA-MG53 sequence; or c) Tet-tPA-MG53 plasmid.

33) (canceled)

34) (canceled)

35) (canceled)

36) (canceled)

37) (canceled)

38) (canceled)

39) (canceled)

40) (canceled)

41) (canceled)

42) (canceled)

43) The viral vector of claim 31, wherein: a) the plasmid comprises the tPA-MG53 sequence cloned behind a CMV promoter; b) the CMV promoter sequence is controllable via the tetracycline (Tet)-response element (TRE), thereby forming Tet-tPA-MG53 plasmid; c) the plasmid further comprises a sequence for SV40-driven transcription of mCherry fluorescent marker; d) the plasmid comprises a promoter DNA sequence preceding the MG53 DNA sequence; e) the plasmid further comprises the TetON DNA sequence preceding the promoter DNA sequence; or f) a combination thereof.

44) (canceled)

45) (canceled)

46) (canceled)

47) (canceled)

48) (canceled)

49) (canceled)

50) (canceled)

51) (canceled)

52) The method of claim 1 comprising: a) administering to a subject in need thereof a viral vector that induces expression of MG53 after administration; or b) administering to a subject in need thereof at least one stem cell comprising a viral vector that induces expression of MG53 in said at least one stem cell.

53) (canceled)

54) (canceled)

55) (canceled)

56) (canceled)

57) The method of claim 1, wherein said dosage forms is selected from the group consisting of: a) bioengineered limbal (limbus) stem cells that express and release MG53; b) viral vector, adenoviral vector, or retroviral vector that enters cellular tissue of the eye or eye socket and causes expression of MG53 in said cellular tissue and release of MG53 from said cellular tissue; c) amniotic membrane or amniotic fluid comprising added exogenous MG53; d) autologous blood serum comprising added exogenous MG53; e) collagen shield comprising added exogenous MG53; f) amniotic membrane or amniotic fluid comprising viral vector, adenoviral vector, or retroviral vector that causes expression of MG53 in cellular tissue; g) amniotic membrane or amniotic fluid comprising bioengineered limbal (limbus) stem cells that express and release MG53; h) autologous blood serum comprising viral vector, adenoviral vector, or retroviral vector that causes expression of MG53 in cellular tissue; h) autologous blood serum comprising bioengineered limbal (limbus) stem cells that express and release MG53; i) collagen shield comprising viral vector, adenoviral vector, or retroviral vector that causes expression of MG53 in cellular tissue; j) collagen shield comprising bioengineered limbal (limbus) stem cells that express and release MG53; and k) a combination of any two or more of the above.

58) The method of claim 1, wherein the dosage form is selected from the group consisting of a liquid, solution, suspension, gel, cream, ointment, implant, explant, slab gel, or coated contact lens.

59) The method of claim 1, wherein the dosage form releases or provides MG53 to the surface of the eye, the corneal surface, the surface of the orbit of the eye, the aqueous humor and/or the vitreous humor.

60) The method of claim 1, wherein the dosage form is administered to the eye, the orbit of the eye, tissue adjacent the eye, topically, intramuscularly, intravenously, subcutaneously, subconjunctivally, systemically, or a combination of two or more thereof.

61) The method of claim 1, wherein the dosage form is administered acutely or chronically.

62) The method of claim 1, wherein the dosage form is administered one, two, three or more times per day.

63) The method of claim 1, wherein the dosage form is administered daily, weekly, monthly, bimonthly, quarterly, semiannually, annually or even longer as needed.

64) The method of claim 1, wherein the dosage form is administered every other day, five times per week, four times per week, three times per week, two times per week, once daily, twice daily, one to four times daily, continuously, or as frequently or infrequently as needed.

65) (canceled)

66) The bioengineered stem cell of claim 9, wherein said viral vector comprises a) plasmid that induces expression of MG53 in stem cells following infection of said stem cells with said viral vector; b) adenovirus comprising a plasmid comprising a tissue plasminogen activator (tPA) leader sequence ahead of a human MG53 cDNA, thereby forming a tPA-MG53 sequence; or c) Tet-tPA-MG53 plasmid.

67) The bioengineered stem cell of claim 66, wherein a) the plasmid comprises the tPA-MG53 sequence cloned behind a CMV promoter; b) the CMV promoter sequence is controllable via the tetracycline (Tet)-response element (TRE), thereby forming Tet-tPA-MG53 plasmid; c) the plasmid further comprises a sequence for SV40-driven transcription of mCherry fluorescent marker; d) the plasmid comprises a promoter DNA sequence preceding the MG53 DNA sequence; e) the plasmid further comprises the TetON DNA sequence preceding the promoter DNA sequence; or f) a combination thereof.
